(19) United States
(12) Patent Application Publication
(10) Pub. No.: US 2026/0294923 A1
(43) Pub. Date: Oct. 1, 2026

(54) COMPOSITIONS AND METHODS FOR TREATING POST-TRAUMATIC STRESS DISORDER

(71) Applicants: Yale University, New Haven, CT (US); The United States Government as Represented by The Department of Veterans Affairs, Washington, DC (US)

(72) Inventors: Alfred Kaye, New Haven, CT (US); Christopher Pittenger, New Haven, CT (US); John Krystal, New Haven, CT (US); Abigail Yu, New Haven, CT (US); Axel F. Rosado, New Haven, CT (US)

(21) Appl. No.: 19/479,201

(22) PCT Filed: Apr. 26, 2024
(86) PCT No.: PCT/US2024/026542

§ 371 Date: Oct. 27, 2025

Related U.S. Application Data
(60) Provisional application No. 63/499,089, filed on Apr. 28, 2023.

(51) Int. Cl. A61K 31/5377; A61K 31/155; A61K 31/343; A61K 31/4045; A61P 25/00

(52) U.S. Cl. CPC A61K 31/5377; A61K 31/155; A61K 31/343; A61K 31/4045; A61P 25/00

(57) ABSTRACT

Provided herein are methods and compositions for treating, ameliorating, or preventing post-traumatic stress disorder (PTSD). The therapeutic methods and compositions described herein include the use of at least one norepinephrine inhibitor and at least one entactogen to treat, ameliorate, or prevent PTSD. The therapeutic methods and compositions described herein also include the use of at least one α_{2A}-adrenergic receptor agonist and at least one psychedelic agent to treat, ameliorate, or prevent PTSD.

### CROSS-REFERENCE TO RELATED APPLICATION

This application claims priority to U.S. Provisional Patent Application No. 63/499,089 entitled “COMPOSITIONS AND METHODS FOR TREATING POST-TRAUMATIC STRESS DISORDER,” filed Apr. 28, 2023, the disclosure of which is incorporated herein by reference in its entirety.

### STATEMENT REGARDING FEDERALLY SPONSORED RESEARCH

This invention was made with government support under MH122733 awarded by the National Institutes of Health. The government has certain rights in the invention.

### BACKGROUND

MDMA (3,4-methylenedioxymethamphetamine) has shown efficacy in Phase 3 clinical trials for PTSD but has several undesirable side-effects, such as, for example, abuse potential (addiction), cardiovascular effects, and neurotoxic effects. In contrast, psychedelic drugs enhance the structural plasticity of prefrontal cortex. Thus, there is a pressing need to develop safe and effective therapies to treat PTSD without negative side effects.

The present disclosure solves this unmet need.

### BRIEF SUMMARY OF THE INVENTION

In one aspect, a method of treating, ameliorating, and/or preventing post-traumatic stress disorder in a subject in need thereof is provided. In certain embodiments, the method includes administering to the subject a pharmaceutical composition comprising a therapeutically effective amount of a norepinephrine inhibitor (NEI), or a pharmaceutically acceptable salt, solvate, enantiomer, diastereomer, or tautomer thereof. In certain embodiments, the method includes administering to the subject a pharmaceutical composition comprising a therapeutically effective amount of an entactogen, or a pharmaceutically acceptable salt, solvate, enantiomer, diastereomer, or tautomer thereof. In certain embodiments, the pharmaceutical composition comprising the NEI and the pharmaceutical composition comprising the entactogen are formulated as a single dosage form or as separate dosage forms. In certain embodiments, the post-traumatic stress disorder in the subject is treated, ameliorated, or prevented.

In another aspect, the method of treating, ameliorating, and/or preventing post-traumatic stress disorder in a subject in need thereof. In certain embodiments, the method includes administering to the subject a pharmaceutical composition comprising a therapeutically effective amount of a α_{2A}-adrenergic receptor agonist (AARA), or a pharmaceutically acceptable salt, solvate, enantiomer, diastereomer, or tautomer thereof. In certain embodiments, the method includes administering to the subject a pharmaceutical composition comprising a therapeutically effective amount of a psychedelic agent or a pharmaceutically acceptable salt, solvate, enantiomer, diastereomer, or tautomer thereof. In certain embodiments, the pharmaceutical composition comprising the AARA and the pharmaceutical composition comprising the psychedelic agent are formulated as a single dosage form or as separate dosage forms. In certain embodiments, the post-traumatic stress disorder in the subject is treated, ameliorated, or prevented.

### BRIEF DESCRIPTION OF THE FIGURES

The drawings illustrate generally, by way of example, but not by way of limitation, various embodiments of the present application.

FIG. 1A depicts a fluorescent biosensor (AAV9-hSyn-GRABNE2h) was injected in the mPFC and a fiber implanted over the region. FIG. 1B shows an example of individual traces of NE (norepinephrine) levels for injections of saline and MDMA. FIG. 1C shows a schematic of an automated detection setup for head twitch behavior for use with a mouse with magnetic ear tag. FIG. 1D shows a schematic of a proposed non-limiting mechanism of MDMA action. Without being bound by theory, MDMA increases release of 5-HT (5-hydroxytryptamine), which acts on 5-HT_{2A} receptors to increase head twitches. MDMA also increases release of NE through binding to the NE transporter. Administration of reboxetine can block the NE transporter, inhibiting the release of NE from MDMA. FIG. 1E shows a left panel with NE levels for injections of MDMA co-administered with reboxetine (n=7) and fluoxetine (n=7). The right panel in FIG. 1E shows the average dF/F between 600-1000 seconds. Paired T test P=0.0173 (*), t=3.500, df=5). FIG. 1F shows a left panel depicting head twitches over 30 minutes with reboxetine (10 mg/kg) or saline administered 1 minute before MDMA (5 mg/kg). Unpaired T test p=0.0001 (***), t=6.069, df=10. The right panel in FIG. 1F shows a time course of head twitches per minute for reboxetine (10 mg/kg) or saline with MDMA (5 mg/kg).

FIGS. 2A-2C show aspects of the methods and compositions described herein, in accordance with various embodiments. FIG. 2A shows a schematic of a proposed non-limiting model of action of MDMA and drug function. Without being bound by theory, it was hypothesized that NE was reducing the head twitch response through the alpha 2 noradrenergic receptor. Yohimbine, an alpha 2 antagonist, will block NE from binding, and the number of head twitches was hypothesized to increase. FIG. 2B shows head twitches over 30 minutes with yohimbine (2 mg/kg) or distilled water administered 10 minutes before MDMA (10 mg/kg). Unpaired T test p=0.0133 (*), t=3.166, df=8. FIG. 2C shows a time course of head twitches per minute for yohimbine (2 mg/kg) or distilled water with MDMA (10 mg/kg).

FIG. 3A shows a schematic of a proposed non-limiting model of action of MDMA and drug function. Without being bound by theory, it was hypothesized that NE was reducing the head twitch response through the alpha 2 noradrenergic receptor. Administration of reboxetine will block NE release from MDMA, but guanfacine, an alpha 2 agonist, will bind to the alpha 2 receptor and inhibit the head twitch response in the absence of NE. FIG. 3B shows head twitches over 30 minutes with guanfacine (0.15 mg/kg) or saline administered 30 minutes before MDMA (10 mg/kg) and reboxetine (10 mg/kg) administered 1 minute before MDMA. Unpaired T test p=0.0016 (**), t=4.283, df=10. FIG. 3C shows a time course of head twitches per minute for guanfacine (0.15 mg/kg) or saline with reboxetine (10 mg/kg) and MDMA (10 mg/kg).

FIG. 4A shows a schematic of a proposed non-limiting model of action of psilocybin and drug function. Psilocybin is an agonist at the 5-HT_{2A} receptor, but guanfacine, an alpha 2 agonist, will bind to the alpha 2 receptor and inhibit the head twitch response in the absence of NE. FIG. 4B shows head twitches over 30 minutes with guanfacine (0.15 mg/kg) or saline administered 30 minutes before psilocybin (1 mg/kg). Unpaired T test p=0.0081 (**), t=3.296, df=10. FIG. 4C shows a time course of head twitches per minute for guanfacine (0.15 mg/kg) or saline with psilocybin (1 mg/kg).

### DETAILED DESCRIPTION

Reference will now be made in detail to certain embodiments of the disclosed subject matter. While the disclosed subject matter will be described in conjunction with the enumerated claims, it will be understood that the exemplified subject matter is not intended to limit the claims to the disclosed subject matter.

Throughout this document, values expressed in a range format should be interpreted in a flexible manner to include not only the numerical values explicitly recited as the limits of the range, but also to include all the individual numerical values or sub-ranges encompassed within that range as if each numerical value and sub-range is explicitly recited. For example, a range of “about 0.1% to about 5%” or “about 0.1% to 5%” should be interpreted to include not just about 0.1% to about 5%, but also the individual values (e.g., 1%, 2%, 3%, and 4%) and the sub-ranges (e.g., 0.1% to 0.5%, 1.1% to 2.2%, 3.3% to 4.4%) within the indicated range. The statement “about X to Y” has the same meaning as “about X to about Y,” unless indicated otherwise. Likewise, the statement “about X, Y, or about Z” has the same meaning as “about X, about Y, or about Z,” unless indicated otherwise.

In this document, the terms “a,” “an,” or “the” are used to include one or more than one unless the context clearly dictates otherwise. The term “or” is used to refer to a nonexclusive “or” unless otherwise indicated. The statement “at least one of A and B” or “at least one of A or B” has the same meaning as “A, B, or A and B.” In addition, it is to be understood that the phraseology or terminology employed herein, and not otherwise defined, is for the purpose of description only and not of limitation. Any use of section headings is intended to aid reading of the document and is not to be interpreted as limiting; information that is relevant to a section heading may occur within or outside of that particular section.

In the methods described herein, the acts can be carried out in any order, except when a temporal or operational sequence is explicitly recited. Furthermore, specified acts can be carried out concurrently unless explicit claim language recites that they be carried out separately. For example, a claimed act of doing X and a claimed act of doing Y can be conducted simultaneously within a single operation, and the resulting process will fall within the literal scope of the claimed process.

### Definitions

The term “about” as used herein can allow for a degree of variability in a value or range, for example, within 10%, within 5%, or within 1% of a stated value or of a stated limit of a range, and includes the exact stated value or range.

The term “substantially” as used herein refers to a majority of, or mostly, as in at least about 50%, 60%, 70%, 80%, 90%, 95%, 96%, 97%, 98%, 99%, 99.5%, 99.9%, 99.99%, or at least about 99.999% or more, or 100%. The term “substantially free of” as used herein can mean having none or having a trivial amount of, such that the amount of material present does not affect the material properties of the composition including the material, such that the composition is about 0 wt % to about 5 wt % of the material, or about 0 wt % to about 1 wt %, or about 5 wt % or less, or less than, equal to, or greater than about 4.5 wt %, 4, 3.5, 3, 2.5, 2, 1.5, 1, 0.9, 0.8, 0.7, 0.6, 0.5, 0.4, 0.3, 0.2, 0.1, 0.01, or about 0.001 wt % or less. The term “substantially free of” can mean having a trivial amount of, such that a composition is about 0 wt % to about 5 wt % of the material, or about 0 wt % to about 1 wt %, or about 5 wt % or less, or less than, equal to, or greater than about 4.5 wt %, 4, 3.5, 3, 2.5, 2, 1.5, 1, 0.9, 0.8, 0.7, 0.6, 0.5, 0.4, 0.3, 0.2, 0.1, 0.01, or about 0.001 wt % or less, or about 0 wt %.

The term “independently selected from” as used herein refers to referenced groups being the same, different, or a mixture thereof, unless the context clearly indicates otherwise. Thus, under this definition, the phrase “X¹, X², and X³ are independently selected from noble gases” would include the scenario where, for example, X¹, X², and X³ are all the same, where X¹, X², and X³ are all different, where X¹ and X² are the same but X³ is different, and other analogous permutations.

The term “room temperature” as used herein refers to a temperature of about 15° C. to 28° C.

The term “standard temperature and pressure” as used herein refers to 20° C. and 101 kPa.

As used herein, the term “composition” or “pharmaceutical composition” refers to a mixture of at least one compound described herein with a pharmaceutically acceptable carrier. The pharmaceutical composition facilitates administration of the compound to a patient or subject. Multiple techniques of administering a compound exist in the art including, but not limited to, intravenous, oral, aerosol, parenteral, ophthalmic, pulmonary and topical administration.

A “disease” is a state of health of an animal wherein the animal cannot maintain homeostasis, and wherein if the disease is not ameliorated then the animal's health continues to deteriorate.

In contrast, a “disorder” in an animal is a state of health in which the animal is able to maintain homeostasis, but in which the animal's state of health is less favorable than it would be in the absence of the disorder. Left untreated, a disorder does not necessarily cause a further decrease in the animal's state of health.

As used herein, the terms “effective amount,” “pharmaceutically effective amount” and “therapeutically effective amount” refer to a nontoxic but sufficient amount of an agent to provide the desired biological result. That result may be reduction and/or alleviation of the signs, symptoms, or causes of a disease, or any other desired alteration of a biological system. An appropriate therapeutic amount in any individual case may be determined by one of ordinary skill in the art using routine experimentation.

As used herein, the term “efficacy” refers to the maximal effect (Emax) achieved within an assay.

As used herein, the term “pharmaceutically acceptable” refers to a material, such as a carrier or diluent, which does not abrogate the biological activity or properties of the compound, and is relatively non-toxic, i.e., the material may be administered to an individual without causing undesirable biological effects or interacting in a deleterious manner with any of the components of the composition in which it is contained.

As used herein, the language “pharmaceutically acceptable salt” refers to a salt of the administered compounds prepared from pharmaceutically acceptable non-toxic acids or bases, including inorganic acids or bases, organic acids or bases, solvates, hydrates, or clathrates thereof.

Suitable pharmaceutically acceptable acid addition salts may be prepared from an inorganic acid or from an organic acid. Examples of inorganic acids include hydrochloric, hydrobromic, hydriodic, nitric, carbonic, sulfuric (including sulfate and hydrogen sulfate), and phosphoric acids (including hydrogen phosphate and dihydrogen phosphate). Appropriate organic acids may be selected from aliphatic, cycloaliphatic, aromatic, araliphatic, heterocyclic, carboxylic and sulfonic classes of organic acids, examples of which include formic, acetic, propionic, succinic, glycolic, gluconic, lactic, malic, tartaric, citric, ascorbic, glucuronic, maleic, malonic, saccharin, fumaric, pyruvic, aspartic, glutamic, benzoic, anthranilic, 4-hydroxybenzoic, phenylacetic, mandelic, embonic (pamoic), methanesulfonic, ethanesulfonic, benzenesulfonic, pantothenic, trifluoromethanesulfonic, 2-hydroxyethanesulfonic, p-toluenesulfonic, sulfanilic, cyclohexylaminosulfonic, stearic, alginic, β-hydroxybutyric, salicylic, galactaric and galacturonic acid.

Suitable pharmaceutically acceptable base addition salts of compounds described herein include, for example, ammonium salts, metallic salts including alkali metal, alkaline earth metal and transition metal salts such as, for example, calcium, magnesium, potassium, sodium and zinc salts. Pharmaceutically acceptable base addition salts also include organic salts made from basic amines such as, for example, N,N′-dibenzylethylene-diamine, chloroprocaine, choline, diethanolamine, ethylenediamine, meglumine (N-methylglucamine) and procaine. All of these salts may be prepared from the corresponding compound by reacting, for example, the appropriate acid or base with the compound.

As used herein, the term “pharmaceutically acceptable carrier” or “pharmaceutically acceptable excipient” means a pharmaceutically acceptable material, composition or carrier, such as a liquid or solid filler, stabilizer, dispersing agent, suspending agent, diluent, excipient, thickening agent, solvent or encapsulating material, involved in carrying or transporting a compound described herein within or to the patient such that it may perform its intended function. Typically, such constructs are carried or transported from one organ, or portion of the body, to another organ, or portion of the body. Each carrier must be “acceptable” in the sense of being compatible with the other ingredients of the formulation, including the compound(s) described herein, and not injurious to the patient. Some examples of materials that may serve as pharmaceutically acceptable carriers include: sugars, such as lactose, glucose and sucrose; starches, such as corn starch and potato starch; cellulose, and its derivatives, such as sodium carboxymethyl cellulose, ethyl cellulose and cellulose acetate; powdered tragacanth; malt; gelatin; talc; excipients, such as cocoa butter and suppository waxes; oils, such as peanut oil, cottonseed oil, safflower oil, sesame oil, olive oil, corn oil and soybean oil; glycols, such as propylene glycol; polyols, such as glycerin, sorbitol, mannitol and polyethylene glycol; esters, such as ethyl oleate and ethyl laurate; agar; buffering agents, such as magnesium hydroxide and aluminum hydroxide; surface active agents; alginic acid; pyrogen-free water; isotonic saline; Ringer's solution; ethyl alcohol; phosphate buffer solutions; and other non-toxic compatible substances employed in pharmaceutical formulations. As used herein, “pharmaceutically acceptable carrier” also includes any and all coatings, antibacterial and antifungal agents, and absorption delaying agents, and the like that are compatible with the activity of the compound(s) described herein, and are physiologically acceptable to the patient. Supplementary active compounds may also be incorporated into the compositions. The “pharmaceutically acceptable carrier” may further include a pharmaceutically acceptable salt of the compound(s) described herein. Other additional ingredients that may be included in the pharmaceutical compositions used with the methods or compounds described herein are known in the art and described, for example in Remington's Pharmaceutical Sciences (Genaro, Ed., Mack Publishing Co., 1985, Easton, PA), which is incorporated herein by reference.

The terms “patient,” “subject,” or “individual” are used interchangeably herein, and refer to any animal, or cells thereof whether in vitro or in situ, amenable to the methods described herein. In a non-limiting embodiment, the patient, subject or individual is a human.

As used herein, the term “potency” refers to the dose needed to produce half the maximal response (ED₅₀).

A “therapeutic” treatment is a treatment administered to a subject who exhibits signs of pathology, for the purpose of diminishing or eliminating those signs.

As used herein, the term “treatment” or “treating” is defined as the application or administration of a therapeutic agent, i.e., a compound or compounds as described herein (alone or in combination with another pharmaceutical agent), to a patient, or application or administration of a therapeutic agent to an isolated tissue or cell line from a patient (e.g., for diagnosis or ex vivo applications), who has a condition contemplated herein or a symptom of a condition contemplated herein, with the purpose to cure, heal, alleviate, relieve, alter, remedy, ameliorate, improve or affect a condition contemplated herein, or the symptoms of a condition contemplated herein. Such treatments may be specifically tailored or modified, based on knowledge obtained from the field of pharmacogenomics.

As used herein, “administered concurrently” means as short a time as possible within the practical physical limits of the ability of the patient taking the dosage form(s) or the practical physical abilities of a medical professional administering the dosage form(s). In some embodiments, “administered concurrently” includes simultaneous administration.

### Therapeutic Compositions

i. Entactogens and NEIs

Provided herein are entactogens useful within the disclosure. Provided herein are norepinephrine inhibitors (NEIs) useful within the disclosure.

Provided herein are therapeutic compositions comprising at least one entactogen and at least one norepinephrine inhibitor (NEI).

Suitable entactogens include, for example, MDMA (3,4-methylenedioxymethamphetamine), MDA (3,4-methylenedioxyamphetamine), MDEA (3,4-methylenedioxy-N-ethylamphetamine), MDOH (3,4-methylenedioxy-N-hydroxyamphetamine), MBDB (1,3-benzodioxolyl-N-methylbutanamine), 5-APB (1-benzofuran-5-ylpropan-2-amine), 5-MAPB (1-(benzofuran-5-yl)-N-methylpropan-2-amine), 6-APB (6-(2-aminopropyl)benzofuran), 6-MAPB (1-(benzofuran-6-yl)-N-methylpropan-2-amine), methylone (3,4-methylenedioxy-N-methylcathinone), mephedrone (4-methylmetheathinone), GHB (γ-hydroxybutyric acid), αMT (α-methyltryptamine), αET (α-ethyltryptamine), MDAI (5,6-methylenedioxy-2-aminoindane), and the like, including solvates, enantiomers, diastereomers, tautomers, and salts (such as pharmaceutically acceptable salts) thereof.

Suitable NEIs include, for example, atomoxetine ((R)—N-methyl-3-phenyl-3-(o-tolyloxy)propan-1-amine), reboxetine (rei-(2R)-2-[(R)-(2-ethoxyphenoxy)(phenyl)methyl]morpholine), viloxazine ((RS)-2-[(2-ethoxyphenoxy)methyl]morpholine), Amedalin (UK-3540-1), Daledalin (UK-3557-15), Edivoxetine (LY-2216684), Esreboxetine (AXS-14; PNU-165442G), Lortalamine (LM-1404), Nisoxetine (LY-94,939), Talopram (tasulopram) (Lu 3-010), Talsupram (Lu 5-005), Tandamine (AY-23,946), and the like, including solvates, enantiomers, diastereomers, tautomers, and salts (such as pharmaceutically acceptable salts) thereof.

The therapeutic compositions can be in separate dosage forms or in a single dosage form. A separate dosage form for the therapeutic composition described herein means that the entactogen is present in a first dosage form administrable by any of the delivery methods described herein, and the NEI is present in a second dosage form administrable by any of the delivery methods described herein. A separate dosage form contains only the entactogen as the active pharmaceutical ingredient or only the NEI as the active pharmaceutical ingredient. Each of the separate dosage forms can further independently include one or more pharmaceutically acceptable carriers or excipients, as described herein. Thus, the pharmaceutically acceptable carriers or excipients in the NEI-containing dosage form can be the same or different as the pharmaceutically acceptable carriers or excipients in the entactogen-containing dosage form.

The dosage form containing the entactogen, either the single dosage form or the separate dosage form, can be formulated as a dosage form with anti-abuse features. Anti-abuse features can include 1) through a prodrug of the entactogen, 2) via an intractable matrix formulation technique, and 3) by antagonist incorporation into the product formulation. In certain embodiments, the anti-abuse properties can include formulation of acid addition salts of the entactogen such that the salt is insoluble in the mucosal membranes of humans, but the entactogen becomes bioavailable when the salt is subjected to the environment in the gastro-intestinal tract. In certain non-limiting embodiments, a dosage form having anti-abuse features can be formulated as described in, for example, U.S. Pat. No. 7,842,307, GB 2238478A, U.S. Pat. No. 7,230,005, WO 2010/044842, WO 2013/003845, EP 1611880, U.S. Pat. Nos. 7,510,726, 7,399,488, US 2009/0215808, US 2010/0249045, WO 2010/105672, WO 2010/066034, U.S. Pat. No. 10,420,729, and the like, the disclosures of which are herein incorporated by reference.

When the dosage form is a single solid dosage form, such as a tablet, the dosage form can be formulated such that the NEI is formulated to be an immediate release component and the entactogen is a delayed or sustained release component. Alternatively, when the dosage form is a single solid dosage form, such as a tablet, the dosage form can be formulated such that the entactogen is formulated to be an immediate release component and the NEI is a delayed or sustained release component. The NEI and entactogen dosage forms, either single dosage forms or separate dosage forms, can be formulated as pharmaceutical compositions containing at least pharmaceutically acceptable excipient or carrier as described herein.

In certain embodiments, the single or separate dosage form contains about 1 to about 1000 mg, or about 1 to about 500 mg, or about 1 to about 400 mg, or about 1 to about 300 mg, or about 1 to about 250 mg, or about 1 to about 200 mg, or about 1 to about 150 mg, or about 1 to about 100 mg, or about 1 to about 90 mg, or about 1 to about 80 mg, or about 1 to about 70 mg, or about 1 to about 60 mg, or about 1 to about 50 mg, or about 1 to about 40 mg, or about 1 to about 30 mg, or about 1 to about 20 mg, or about 1 to about 10 mg of the entactogen, or a solvate, enantiomer, diastereomer, tautomer, or pharmaceutically acceptable salt thereof. In certain embodiments, the single or separate dosage form contains about 5, 10, 15, 20, 25, 30, 35, 40, 45, 50, 55, 60, 65, 70, 75, 80, 85, 90, 95, 100, 105, 110, 115, 120, 125, 130, 135, 140, 145, 150, 155, 160, 165, 170, 175, 180, 185, 190, 195, 200, 205, 210, 215, 220, 225, 230, 235, 240, 245, 250, 255, 260, 265, 270, 275, 280, 285, 290, 295, 300, 305, 310, 315, 320, 325, 330, 335, 340, 345, 350, 355, 360, 365, 370, 375, 380, 385, 390, 395, 400, 405, 410, 415, 420, 425, 430, 435, 440, 445, 450, 455, 460, 465, 470, 475, 480, 485, 490, 495, 500, 505, 510, 515, 520, 525, 530, 535, 540, 545, 550, 555, 560, 565, 570, 575, 580, 585, 590, 595, 600, 605, 610, 615, 620, 625, 630, 635, 640, 645, 650, 655, 660, 665, 670, 675, 680, 685, 690, 695, 700, 705, 710, 715, 720, 725, 730, 735, 740, 745, 750, 755, 760, 765, 770, 775, 780, 785, 790, 795, 800, 805, 810, 815, 820, 825, 830, 835, 840, 845, 850, 855, 860, 865, 870, 875, 880, 885, 890, 895, 900, 905, 910, 915, 920, 925, 930, 935, 940, 945, 950, 955, 960, 965, 970, 975, 980, 985, 990, 995, or 1000 mg of the entactogen, or a solvate, enantiomer, diastereomer, tautomer, or pharmaceutically acceptable salt thereof.

In certain embodiments, the single or separate dosage form contains about 100, 105, 110, 115, 102, 125, 130, 135, or 150 mg of the entactogen, or a solvate, enantiomer, diastereomer, tautomer, or pharmaceutically acceptable salt thereof. In certain embodiments, the single or separate dosage form contains about 125 mg of MDMA.

In certain embodiments, the single or separate dosage form contains about 1 to about 1000 mg, or about 1 to about 500 mg, or about 1 to about 400 mg, or about 1 to about 300 mg, or about 1 to about 250 mg, or about 1 to about 200 mg, or about 1 to about 150 mg, or about 1 to about 100 mg, or about 1 to about 90 mg, or about 1 to about 80 mg, or about 1 to about 70 mg, or about 1 to about 60 mg, or about 1 to about 50 mg, or about 1 to about 40 mg, or about 1 to about 30 mg, or about 1 to about 20 mg, or about 1 to about 10 mg of the NEI, or a solvate, enantiomer, diastereomer, tautomer, or pharmaceutically acceptable salt thereof. In certain embodiments, the single or separate dosage form contains about 1, 2, 3, 4, 5, 6, 7, 8, 9, 10, 15, 20, 25, 30, 35, 40, 45, 50, 55, 60, 65, 70, 75, 80, 85, 90, 95, 100, 105, 110, 115, 120, 125, 130, 135, 140, 145, 150, 155, 160, 165, 170, 175, 180, 185, 190, 195, 200, 205, 210, 215, 220, 225, 230, 235, 240, 245, 250, 255, 260, 265, 270, 275, 280, 285, 290, 295, 300, 305, 310, 315, 320, 325, 330, 335, 340, 345, 350, 355, 360, 365, 370, 375, 380, 385, 390, 395, 400, 405, 410, 415, 420, 425, 430, 435, 440, 445, 450, 455, 460, 465, 470, 475, 480, 485, 490, 495, 500, 505, 510, 515, 520, 525, 530, 535, 540, 545, 550, 555, 560, 565, 570, 575, 580, 585, 590, 595, 600, 605, 610, 615, 620, 625, 630, 635, 640, 645, 650, 655, 660, 665, 670, 675, 680, 685, 690, 695, 700, 705, 710, 715, 720, 725, 730, 735, 740, 745, 750, 755, 760, 765, 770, 775, 780, 785, 790, 795, 800, 805, 810, 815, 820, 825, 830, 835, 840, 845, 850, 855, 860, 865, 870, 875, 880, 885, 890, 895, 900, 905, 910, 915, 920, 925, 930, 935, 940, 945, 950, 955, 960, 965, 970, 975, 980, 985, 990, 995, or 1000 mg of the NEI, or a solvate, enantiomer, diastereomer, tautomer, or pharmaceutically acceptable salt thereof.

In certain embodiments, the single or separate dosage form contains about 1, 2, 3, 4, 5, 6, 7, 8, 9, 10, 11, 12, 13,14, 15, 16, 17, 18, 19, or 20 mg of the NEI, or a solvate, enantiomer, diastereomer, tautomer, or pharmaceutically acceptable salt thereof. In certain embodiments, the single or separate dosage form contains 8 mg of reboxetine.

In certain embodiments, the NEI is administered prior to administration of the entactogen. In certain embodiments, the NEI can be administered about 1, 5, 10, 15, 30, or 60 minutes before administration of the entactogen. In certain embodiments, the NEI can be administered about 2, 3, 4, 5, 6, 7, 8, 9, 10, 11, 12, 13, 14, 15, 16, 17, 18, 19, 20, 21, 22, 23, or 24 hours before administration of the entactogen. In certain embodiments, the NEI and the entactogen are administered concurrently, for example either as part of a single dosage form, or as part of separate dosage forms taken together at the same time.

In certain embodiments, the entactogen is MDMA.

In certain embodiments, the NEI is reboxetine.

ii. Psychedelic Agents and α_{2A}-Adrenergic Receptor Agonists

Provided herein are psychedelic agents useful within the disclosure. Provided herein are α_{2A}-adrenergic receptor agonists (AARAs) useful within the disclosure.

Provided herein are therapeutic compositions comprising at least one psychedelic agent and at least one α_{2A}-adrenergic receptor agonist (AARA).

Suitable psychedelic agents include, for example, psilocybin, 2C—B (4-bromo-2,5-dimethoxyphenethylamine), ketamine (racemic, R-ketamine, or S-ketamine), DMT (N,N-dimethyltryptamine), 5-MeO-DMT (5-methoxy-dimethyltryptamine), LSA (d-lysergic acid amide), LSD (lysergic acid diethylamide), and the like, including solvates, enantiomers, diastereomers, tautomers, and salts (such as pharmaceutically acceptable salts) thereof. The psilocybin is, in certain embodiments, isolated pure psylocibin that is substantially free of any other substances found in Psilocybe mushroom species. The psilocybin can be at least 95, 96, 97, 98, 99, or 99.9% pure psilocybin.

Suitable AARAs include, for example, clonidine, dexmedetomidine, fadolimidine, guanfacine, guanabenz, guanoxabenz, guanethidine, xylazine, tizanidine, methyldopa, methylnorepinephrine, norepinephrine, detomidine, lofexidine, and the like, including solvates, enantiomers, diastereomers, tautomers, and salts (such as pharmaceutically acceptable salts) thereof.

The therapeutic compositions can be in separate dosage forms or in a single dosage form. A separate dosage form for the therapeutic composition described herein means that the psychedelic agent is present in a first dosage form administrable by any of the delivery methods described herein, and the AARA is present in a second dosage form administrable by any of the delivery methods described herein. A separate dosage form contains only the psychedelic agent as the active pharmaceutically active ingredient or only the AARA as the active pharmaceutically active ingredient. Each of the separate dosage forms can further independently include one or more pharmaceutically acceptable carriers or excipients, as described herein. Thus, the pharmaceutically acceptable carriers or excipients in the AARA-containing dosage form can be the same or different as the pharmaceutically acceptable carriers or excipients in the psychedelic agent-containing dosage form.

The dosage form containing the psychedelic agent, either the single dosage form or the separate dosage form, can be formulated as a dosage form with anti-abuse features. Anti-abuse features can include 1) through a prodrug of the psychedelic agent, 2) via an intractable matrix formulation technique, and 3) by antagonist incorporation into the product formulation. In certain embodiments, the anti-abuse properties can include formulation of acid addition salts of the psychedelic agent such that the salt is insoluble in the mucosal membranes of humans, but the psychedelic agent becomes bioavailable when the salt is subjected to the environment in the gastro-intestinal tract. In certain non-limiting embodiments, a dosage form having anti-abuse features can be formulated as described in, for example, U.S. Pat. No. 7,842,307, GB 2238478A, U.S. Pat. No. 7,230,005, WO 2010/044842, WO 2013/003845, EP 1611880, U.S. Pat. Nos. 7,510,726, 7,399,488, US 2009/0215808, US 2010/0249045, WO 2010/105672, WO 2010/066034, U.S. Pat. No. 10,420,729, and the like, the disclosures of which are herein incorporated by reference.

When the dosage form is a single solid dosage form, such as a tablet, the dosage form can be formulated such that the AARA is formulated to be an immediate release component and the psychedelic agent is a delayed or sustained release component. Alternatively, the when the dosage form is a single solid dosage form, such as a tablet, the dosage form can be formulated such that the psychedelic agent is formulated to be an immediate release component and the AARA is a delayed or sustained release component. The AARA and psychedelic dosage forms, either single dosage forms or separate dosage forms, can be formulated as pharmaceutical compositions containing at least pharmaceutically acceptable excipient or carrier as described herein.

In certain embodiments, the single or separate dosage form contains about 1 to about 1000 mg, or about 1 to about 500 mg, or about 1 to about 400 mg, or about 1 to about 300 mg, or about 1 to about 250 mg, or about 1 to about 200 mg, or about 1 to about 150 mg, or about 1 to about 100 mg, or about 1 to about 90 mg, or about 1 to about 80 mg, or about 1 to about 70 mg, or about 1 to about 60 mg, or about 1 to about 50 mg, or about 1 to about 40 mg, or about 1 to about 30 mg, or about 1 to about 20 mg, or about 1 to about 10 mg of the psychedelic agent, or a solvate, enantiomer, diastereomer, tautomer, or pharmaceutically acceptable salt thereof. In certain embodiments, the single or separate dosage form contains about 5, 10, 15, 20, 25, 30, 35, 40, 45, 50, 55, 60, 65, 70, 75, 80, 85, 90, 95, 100, 105, 110, 115, 120, 125, 130, 135, 140, 145, 150, 155, 160, 165, 170, 175, 180, 185, 190, 195,200, 205, 210, 215, 220, 225, 230, 235, 240, 245, 250, 255, 260, 265, 270, 275, 280, 285, 290, 295, 300, 305, 310, 315, 320, 325, 330, 335, 340, 345, 350, 355, 360, 365, 370, 375, 380, 385, 390, 395, 400, 405, 410, 415, 420, 425, 430, 435, 440, 445, 450, 455, 460, 465, 470, 475, 480, 485, 490, 495, 500, 505, 510, 515, 520, 525, 530, 535, 540, 545, 550, 555, 560, 565, 570, 575, 580, 585, 590, 595, 600, 605, 610, 615, 620, 625, 630, 635, 640, 645, 650, 655, 660, 665, 670, 675, 680, 685, 690, 695, 700, 705, 710, 715, 720, 725, 730, 735, 740, 745, 750, 755, 760, 765, 770, 775, 780, 785, 790, 795, 800, 805, 810, 815, 820, 825, 830, 835, 840, 845, 850, 855, 860, 865, 870, 875, 880, 885, 890, 895, 900, 905, 910, 915, 920, 925, 930, 935, 940, 945, 950, 955, 960, 965, 970, 975, 980, 985, 990, 995, or 1000 mg of the psychedelic agent, or a solvate, enantiomer, diastereomer, tautomer, or pharmaceutically acceptable salt thereof.

In certain embodiments, the single or separate dosage form contains about 5, 10, 15, 20, 25, 30, 35, or 40 mg of the psychedelic agent, or a solvate, enantiomer, diastereomer, tautomer, or pharmaceutically acceptable salt thereof. In certain embodiments, the single or separate dosage form contains about 25 mg of psilocybin, or a solvate, enantiomer, diastereomer, tautomer, or pharmaceutically acceptable salt thereof.

In certain embodiments, the single or separate dosage form contains about 1 to about 1000 mg, or about 1 to about 500 mg, or about 1 to about 400 mg, or about 1 to about 300 mg, or about 1 to about 250 mg, or about 1 to about 200 mg, or about 1 to about 150 mg, or about 1 to about 100 mg, or about 1 to about 90 mg, or about 1 to about 80 mg, or about 1 to about 70 mg, or about 1 to about 60 mg, or about 1 to about 50 mg, or about 1 to about 40 mg, or about 1 to about 30 mg, or about 1 to about 20 mg, or about 1 to about 10 mg of the AARA, or a solvate, enantiomer, diastereomer, tautomer, or pharmaceutically acceptable salt thereof. In certain embodiments, the single or separate dosage form contains about 0.1, 0.5, 1, 2, 3, 4, 5, 6, 7, 9, 10, 15, 20, 25, 30, 35, 40, 45, 50, 55, 60, 65, 70, 75, 80, 85, 90, 95, 100, 105, 110, 115, 120, 125, 130, 135, 140, 145, 150, 155, 160, 165, 170, 175, 180, 185, 190, 195, 200, 205, 210, 215, 220, 225, 230, 235, 240, 245, 250, 255, 260, 265, 270, 275, 280, 285, 290, 295, 300, 305, 310, 315, 320, 325, 330, 335, 340, 345, 350, 355, 360, 365, 370, 375, 380, 385, 390, 395, 400, 405, 410, 415, 420, 425, 430, 435, 440, 445, 450, 455, 460, 465, 470, 475, 480, 485, 490, 495, 500, 505, 510, 515, 520, 525, 530, 535, 540, 545, 550, 555, 560, 565, 570, 575, 580, 585, 590, 595, 600, 605, 610, 615, 620, 625, 630, 635, 640, 645, 650, 655, 660, 665, 670, 675, 680, 685, 690, 695, 700, 705, 710, 715, 720, 725, 730, 735, 740, 745, 750, 755, 760, 765, 770, 775, 780, 785, 790, 795, 800, 805, 810, 815, 820, 825, 830, 835, 840, 845, 850, 855, 860, 865, 870, 875, 880, 885, 890, 895, 900, 905, 910, 915, 920, 925, 930, 935, 940, 945, 950, 955, 960, 965, 970, 975, 980, 985, 990, 995, or 1000 mg of the AARA, or a solvate, enantiomer, diastereomer, tautomer, or pharmaceutically acceptable salt thereof.

In certain embodiments, the single or separate dosage form contains about 0.1, 0.5, 1, 2, 3, 4, 5, 6, 7, 9, or 10 mg of the AARA, or a solvate, enantiomer, diastereomer, tautomer, or pharmaceutically acceptable salt thereof. In certain embodiments, the single or separate dosage form contains about 1 to about 4 mg of guanfacine.

In certain embodiments, the AARA is administered prior to or after administration of the psychedelic agent. In certain embodiments, the AARA can be administered about 1, 5, 10, 15, 30, or 60 minutes before administration of the psychedelic agent. In certain embodiments, the AARA can be administered about 1, 5, 10, 15, 30, or 60 minutes after administration of the psychedelic agent. In certain embodiments, the AARA can be administered about 2, 3, 4, 5, 6, 7, 8, 9, 10, 11, 12, 13, 14, 15, 16, 17, 18, 19, 20, 21, 22, 23, or 24 hours before administration of the psychedelic agent. In certain embodiments, the AARA can be administered about 2, 3, 4, 5, 6, 7, 8, 9, 10, 11, 12, 13, 14, 15, 16, 17, 18, 19, 20, 21, 22, 23, or 24 hours after administration of the psychedelic agent. In certain embodiments, the AARA and the psychedelic agent are administered concurrently, for example either as part of a single dosage form, or as part of separate dosage forms taken together at the same time.

In certain embodiments, the psychedelic agent is psilocybin.

In certain embodiments, the AARA is guanfacine.

The compositions containing the compound(s) described herein include a pharmaceutical composition comprising at least one compound as described herein and at least one pharmaceutically acceptable carrier. In certain embodiments, the composition is formulated for an administration route such as oral or parenteral, for example, transdermal, transmucosal (e.g., sublingual, lingual, (trans)buccal, (trans)urethral, vaginal (e.g., trans- and perivaginally), (intra)nasal and (trans)rectal, intravesical, intrapulmonary, intraduodenal, intragastrical, intrathecal, subcutaneous, intramuscular, intradermal, intra-arterial, intravenous, intrabronchial, inhalation, and topical administration.

### Non-Limiting Discussion

MDMA is currently in Phase 3 clinical trials to treat post-traumatic stress disorder (PTSD) and appears to show substantial efficacy in the reduction of PTSD symptoms. In certain embodiments, MDMA can augment psychotherapy for PTSD by increasing attention to traumatic memories or by strengthening extinction learning. PTSD is characterized by changes in learning and, in this context, enhancement of extinction learning may improve PTSD treatments. In mice, fear extinction enhancement studies show MDMA to enhance extinction of auditory fear associations through serotonin (5-HT) release, and this effect can be recapitulated via direct infusion of MDMA into either medial prefrontal cortex (mPFC) or amygdala.

MDMA enables patients to revisit traumatic memories with greater ease, leading it to be called an “entactogen” (“going within to bring out”). Drug discrimination studies show that animals distinguish between entactogens and classic psychedelics (5-HT_{2A} agonists), and human subjective effects of entactogens are also distinct from classic psychedelics. Mechanistically, entactogens act like stimulants to release norepinephrine (NE) and dopamine (DA) through monoamine transporters, but differ from stimulants in that they also release 5-HT. Within the entactogen class, there is substantial diversity of in vitro selectivity for monoamine transporters. The link between in vivo monoamine-releasing effects of MDMA and specific mechanistic effects relevant to therapeutic plasticity remains unclear.

Pharmacological dissociation studies have established individual monoamines' role in MDMA's behavioral effects. 5-HT release leads to pro-social behavior through 5-HT_{1B} activation in nucleus accumbens and fear extinction enhancement through the 5-HT_{2A} receptor. The rewarding and addictive effects of MDMA are mediated by dopamine release in nucleus accumbens. Locomotor increase by MDMA involves the noradrenergic α₁ receptor in rodents and in humans, NE release contributes to euphoria and cardiovascular activation. Further delineating receptor-dependent effects of MDMA may allow the development of entactogens with improved efficacy in PTSD and tolerability.

Different entactogens were investigated for induction of fear extinction behavior, 5-HT and NE release, and 5-HT_{2A} dependent behavior. Fear extinction was systematically compared across entactogens to determine if other drugs in this class share putative therapeutic effects with MDMA. To characterize neuromodulator effects, in vivo neurotransmitter release measurements and behavior was compared across entactogens and psychedelic drugs. Pharmacological manipulations revealed a previously unreported role for NE in interacting with 5-HT_{2A} agonism in the behavioral effects of MDMA.

Since MDMA is hypothesized to act as a treatment for PTSD by fear extinction enhancement, fear extinction enhancement to auditory cues was assessed using a three-day paradigm to compare entactogens. To determine the distinct roles of 5-HT and NE release in entactogen effects, in vivo behavioral and physiological approaches were used to detangle distinct neuromodulatory release properties between entactogens. In vivo 5-HT_{2A} receptor activity can be identified using the head twitch response (HTR), a sudden rapid movement of the head which occurs with hallucinogenic 5-HT_{2A} agonists and indirectly by drugs that induce serotonin release. An automated magnetometer HTR assay was used to assess 5-HT_{2A} activity of entactogen drugs. Furthermore, fiber photometry and fluorescent GPCR-based sensors for 5-HT and NE were used to separately identify dynamics of neuromodulator release after drug injections. This approach enables high temporal resolution and high specificity measurement of neurotransmitter release, allowing for precise observation of neuromodulator levels after injection of entactogens.

### NE Releasing Effects of Entactogens and HTR

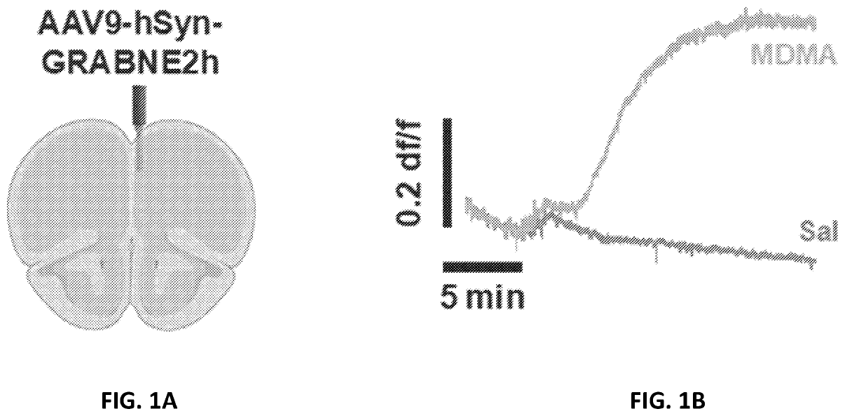

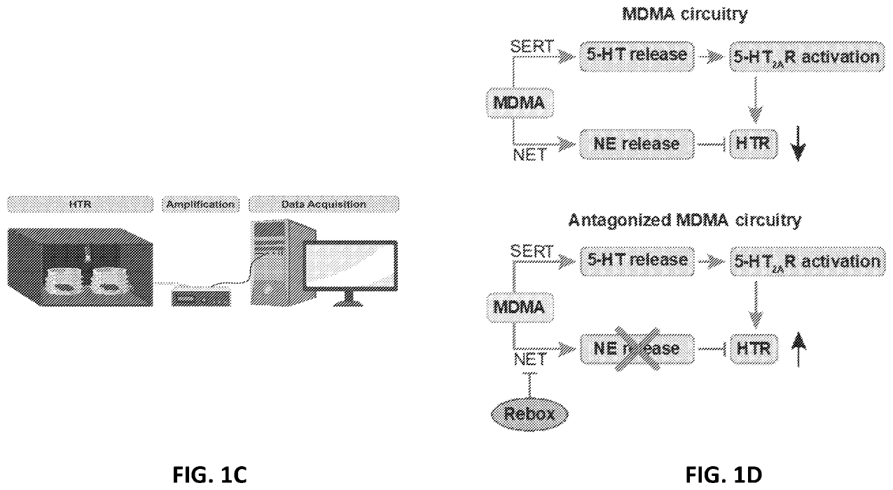

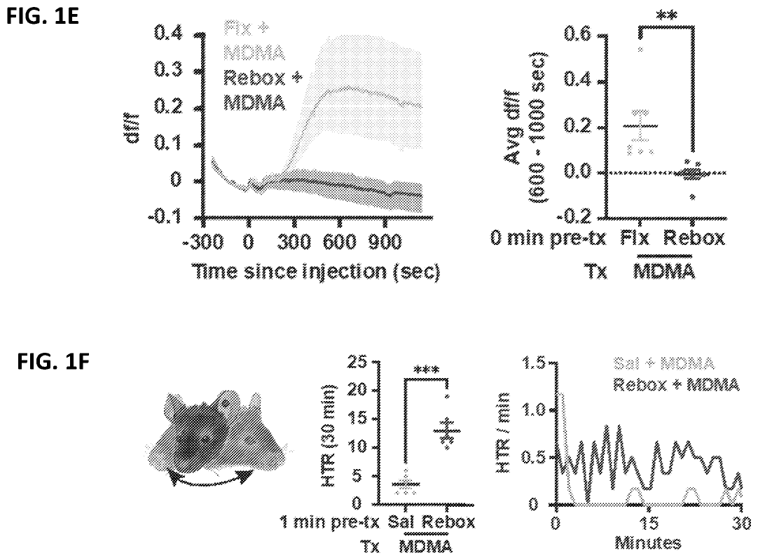

NE release dynamics were determined, since in vitro transporter affinity studies predict differences in NE release properties across entactogens. Using fiber photometry of GPCR based sensors, in vivo NE dynamics in the mPFC (FIG. 1A) were measured with high temporal resolution after administration of entactogens. The physical manipulation involved in intraperitoneal injections evokes a transient increase in NE (FIG. 1B).

Without being bound by theory, it was hypothesized that NE can suppress the head twitch response of entactogens (FIG. 1C-D). Since entactogens induce NE release via presynaptic NE transporters (NET), reboxetine (a NET inhibitor) was used (1 minute pretreatment; reboxetine+MDMA) to block NE release from MDMA. It was confirmed that co-administration of reboxetine one minute before MDMA injection suppresses NE release using fiber photometry of the NE GRAB sensor (GRAB-NE2h, FIG. 1E). This effect was selective: NET inhibition (reboxetine, 10 mg/kg, n=7) but not SERT inhibition (fluoxetine, 10 mg/kg, n=7) suppressed NE release (FIG. 1E).

Saline+MDMA and reboxetine+MDMA were compared to determine whether NE release is responsible for the low HTR behavior of MDMA. In agreement with this hypothesis, reboxetine+MDMA elicited a greater number of head twitches than saline+MDMA (30 minutes; FIG. 1F). Temporally, the head twitch increases in the reboxetine+MDMA group were sustained for at least 30 minutes (FIG. 1F right). Without being bound by theory, it is believed that lower NE leads to higher head twitches for MDMA.

### NE Blockade of HTR Mediated Through the α₂ Adrenergic Receptor

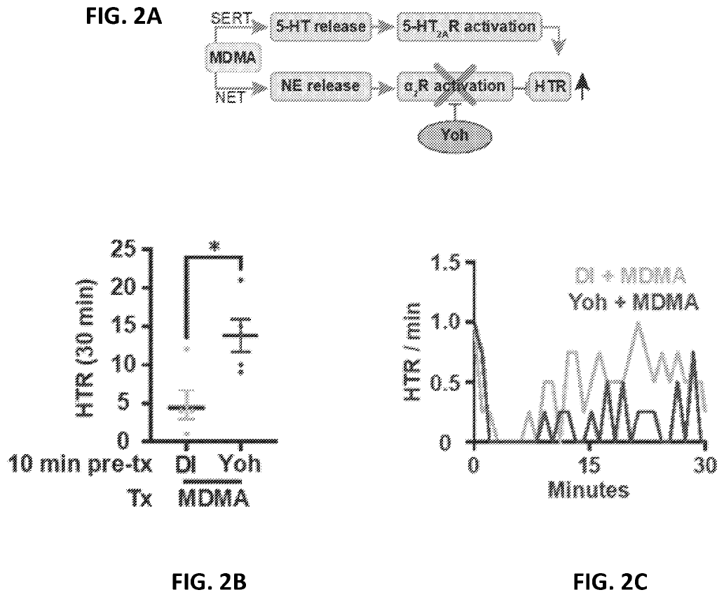

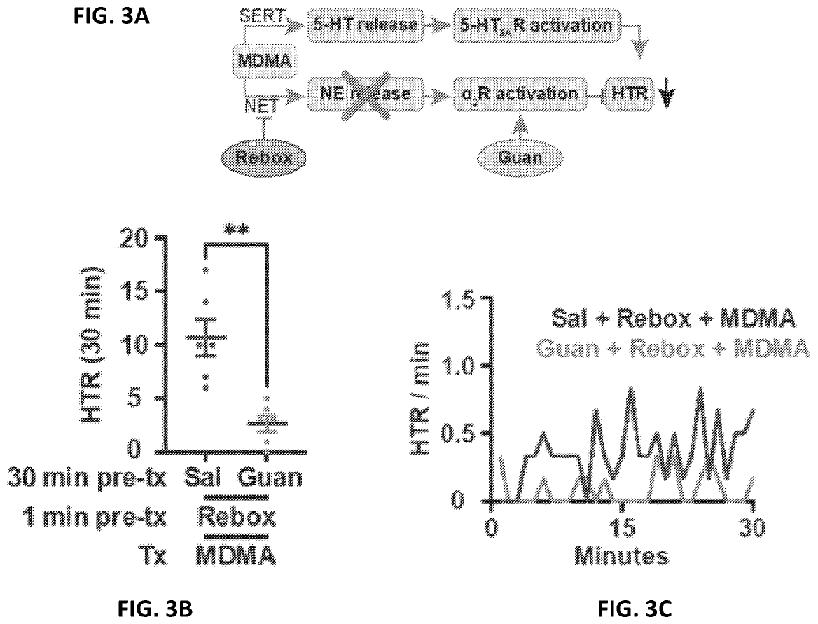

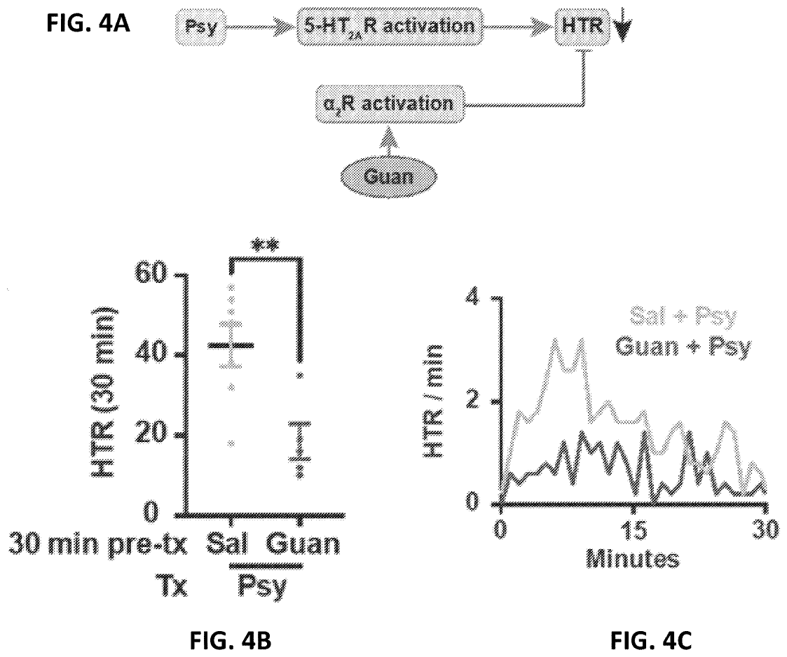

An a₂ antagonist yohimbine (2 mg/kg, n=5) was administered before MDMA (yohimbine+MDMA; FIGS. 2A-2C). Indeed, yohimbine+MDMA increased the HTR relative to vehicle+MDMA (FIGS. 3A-3C, Unpaired T test p=0.0133, t=3.166, df=8). Like reboxetine, suppression of the noradrenergic blocking effect was most pronounced after the initial administration and sustained for 30 minutes (FIGS. 4A-4C).

Since blocking the α₂ receptor unmasked MDMA's 5-HT_{2A} effect on head twitches, activation of this receptor with guanfacine (FIGS. 3A-3C) was undertaken. Previously, it was found that blocking NE release from MDMA with reboxetine led to more head twitches. If the suppressive effect of NE is mediated by the α₂ receptor, then adding guanfacine (MDMA+reboxetine+guanfacine) should reduce head twitches. In this new cohort (MDMA+reboxetine+/−guanfacine, 0.15 mg/kg), a₂ agonism suppressed head twitches (FIG. 3B).

To better understand the implications of α₂-5-HT_{2A} receptor interactions in behavior, guanfacine was tested against psilocybin, a 5-HT_{2A} receptor agonist with more robust HTR (FIGS. 4A-4C). Indeed, guanfacine could suppress the HTR of psilocybin (psilocybin+guanfacine vs. psilocybin+saline (n=7; FIG. 4B) and prevented the previously identified temporal peak. The noradrenergic α₂ NE receptor bidirectionally modulates the 5-HT_{2A} effects of MDMA and can also suppress those effects for a classic psychedelic.

It was found that MDMA's supraphysiological NE release opposes the head twitch behavior (a downstream effect of 5-HT_{2A} agonism) through a₂ noradrenergic receptor activation. Thus, this study uncovered a role for NE in opposing indirect 5-HT_{2A} effects.

Despite observing that NE suppresses the head twitch response, the specific neural circuits subserving this opponency have not been identified. Local infusion of 5-HT_{2A} agonists into the mPFC can induce the HTR, however the specificity and circuit basis of this phenomenon remain unknown. Head twitches also coincide with apparent widespread activation of cortical and hippocampal circuits. NE release by MDMA acts on the α₂ receptor to oppose 5-HT_{2A} agonist effects—intriguingly, the α₂ receptor has well characterized effects on layer V pyramidal cells in the mPFC, which also have been suggested as mediating 5-HT_{2A} effects. Noradrenergic effects of MDMA have also been identified as contributing to subjective euphoria from MDMA in humans and increased locomotion in mice. Circuit-specific recordings and manipulations of the NE pathway may further delineate how the distinct monoaminergic effects of MDMA interact with one another.

Using fluorescent GRAB sensors and fiber photometry provided important insight into the in vivo mechanisms of entactogens. The combination of high temporal resolution and molecular specificity permitted us to make inferences about neuromodulatory release properties of entactogens. This approach was utilized to observe the precise manipulation of NE release by MDMA and NET blockade. These sensors permit spatial and cell-type localization in combination with genetically modified animals, which can be used to further demarcate specific regional differences in entactogen induced monoamine release and identify the downstream circuits affected by drug administration.

5-HT_{2A} agonism plays a key role in generating structural plasticity after psychedelics and may also contribute to fear extinction enhancement. An important caveat is that MDMA also induces social behaviors in mice and humans—enhancing prosocial behavior and trust could also influence psychotherapy efficacy. Since MDMA-assisted psychotherapy may become a critically needed treatment for PTSD, understanding the mechanism of this therapeutic effect is imperative. The results herein point to distinct, surprising, and unexpected neuromodulatory roles of NE and 5-HT in MDMA's effects.

The compounds described herein can possess one or more stereocenters, and each stereocenter can exist independently in either the (R) or (S) configuration. In certain embodiments, compounds described herein are present in optically active or racemic forms. It is to be understood that the compounds described herein encompass racemic, optically-active, regioisomeric and stereoisomeric forms, or combinations thereof that possess the therapeutically useful properties described herein. Preparation of optically active forms is achieved in any suitable manner, including by way of non-limiting example, by resolution of the racemic form with recrystallization techniques, synthesis from optically-active starting materials, chiral synthesis, or chromatographic separation using a chiral stationary phase. In certain embodiments, a mixture of one or more isomer is utilized as the therapeutic compound described herein. In other embodiments, compounds described herein contain one or more chiral centers. These compounds are prepared by any means, including stereoselective synthesis, enantioselective synthesis and/or separation of a mixture of enantiomers and/or diastereomers. Resolution of compounds and isomers thereof is achieved by any means including, by way of non-limiting example, chemical processes, enzymatic processes, fractional crystallization, distillation, and chromatography.

The methods and formulations described herein include the use of N-oxides (if appropriate), crystalline forms (also known as polymorphs), solvates, amorphous phases, and/or pharmaceutically acceptable salts of compounds having the structure of any compound(s) described herein, as well as metabolites and active metabolites of these compounds having the same type of activity. Solvates include water, ether (e.g., tetrahydrofuran, methyl tert-butyl ether) or alcohol (e.g., ethanol) solvates, acetates and the like. In certain embodiments, the compounds described herein exist in solvated forms with pharmaceutically acceptable solvents such as water, and ethanol. In other embodiments, the compounds described herein exist in unsolvated form.

In certain embodiments, the compound(s) described herein can exist as tautomers. All tautomers are included within the scope of the compounds presented herein.

In certain embodiments, compounds described herein are prepared as prodrugs. A “prodrug” refers to an agent that is converted into the parent drug in vivo. In certain embodiments, upon in vivo administration, a prodrug is chemically converted to the biologically, pharmaceutically or therapeutically active form of the compound. In other embodiments, a prodrug is enzymatically metabolized by one or more steps or processes to the biologically, pharmaceutically or therapeutically active form of the compound.

In certain embodiments, sites on, for example, the aromatic ring portion of compound(s) described herein are susceptible to various metabolic reactions. Incorporation of appropriate substituents on the aromatic ring structures may reduce, minimize or eliminate this metabolic pathway. In certain embodiments, the appropriate substituent to decrease or eliminate the susceptibility of the aromatic ring to metabolic reactions is, by way of example only, a deuterium, a halogen, or an alkyl group.

Methods of Treating, Ameliorating, and/or Preventing

The disclosure includes a method of treating, ameliorating, and/or preventing post-traumatic stress disorder (PTSD) using the compounds and/or compositions described herein. The methods described herein also serve to treat, ameliorate, and/or prevent at least one symptom of PTSD. Symptoms of PTSD include, but are not limited to, feeling upset by things that remind a person of what happened, having nightmares, vivid memories, or flashbacks of the event that make a person feel like the traumatic event is happening all over again, feeling emotionally cut off from others, feeling numb or losing interest in things you used to care about, feeling constantly on guard, feeling irritated or having angry outbursts, having difficulty sleeping, having trouble concentrating, being jumpy or easily startled, frequently avoiding places or things that remind a person of what happened, using alcohol and/or drugs to numb feelings, consider harming yourself or others, working all the time to occupy the mind, pulling away from other people and become isolated, and the like. In certain embodiments, the methods described herein also serve to treat, ameliorate, and/or prevent at least two, three, four, five, or more symptoms of PTSD. The methods described herein can be used in administering any of the doses of the entactogen, NEI, psychedelic agent, or AARA described herein.

In certain embodiments, a method of treating, ameliorating, and/or preventing post-traumatic stress disorder in a subject in need thereof is provided. In certain embodiments, the method includes administering to the subject a therapeutically effective amount of at least one norepinephrine inhibitor (NEI), or a solvate, enantiomer, diastereomer, tautomer, or (pharmaceutically acceptable) salt thereof. In certain embodiments, the method includes administering to the subject a therapeutically effective amount of at least one entactogen, or a solvate, enantiomer, diastereomer, tautomer, or (pharmaceutically acceptable) salt thereof. In certain embodiments, the post-traumatic stress disorder in the subject is treated, ameliorated, or prevented.

In certain embodiments, a method of treating, ameliorating, and/or preventing post-traumatic stress disorder in a subject in need thereof. In certain embodiments, the method includes administering to the subject a pharmaceutical composition comprising a therapeutically effective amount of a norepinephrine inhibitor (NEI), or a pharmaceutically acceptable salt, solvate, enantiomer, diastereomer, or tautomer thereof. In certain embodiments, the method includes administering to the subject a pharmaceutical composition comprising a therapeutically effective amount of an entactogen, or a pharmaceutically acceptable salt, solvate, enantiomer, diastereomer, or tautomer thereof. In certain embodiments, the pharmaceutical composition comprising the NEI and the pharmaceutical composition comprising the entactogen are formulated as a single dosage form or as separate dosage forms. In certain embodiments, the post-traumatic stress disorder in the subject is treated, ameliorated, or prevented.

In certain embodiments, the NEI, or a solvate, enantiomer, diastereomer, tautomer, or (pharmaceutically acceptable) salt thereof, is selected from the group consisting of atomoxetine ((R)—N-Methyl-3-phenyl-3-(o-tolyloxy)propan-1-amine), reboxetine (rel-(2R)-2-[(R)-(2-ethoxyphenoxy)(phenyl)methyl]morpholine), viloxazine ((RS)-2-[(2-ethoxyphenoxy)methyl]morpholine), amedalin (UK-3540-1), Daledalin (UK-3557-15), edivoxetine (LY-2216684), esreboxetine (AXS-14; PNU-165442G), lortalamine (LM-1404), nisoxetine (LY-94,939), talopram (tasulopram) (Lu 3-010), talsupram (Lu 5-005), and tandamine (AY-23,946).

In certain embodiments, the entactogen, or a solvate, enantiomer, diastereomer, tautomer, or (pharmaceutically acceptable) salt thereof, is selected from the group consisting of MDMA (3,4-methylenedioxymethamphetamine), MDA (3,4-methylenedioxyamphetamine), MDEA (3,4-methylenedioxy-N-ethylamphetamine), MDOH (3,4-methylenedioxy-N-hydroxyamphetamine), MBDB (1,3-benzodioxolyl-N-methylbutanamine), 5-APB (1-benzofuran-5-ylpropan-2-amine), 5-MAPB (1-(benzofuran-5-yl)-N-methylpropan-2-amine), 6-APB (6-(2-aminopropyl)benzofuran), 6-MAPB (1-(benzofuran-6-yl)-N-methylpropan-2-amine), methylone (3,4-methylenedioxy-N-methylcathinone), mephedrone (4-methylmethcathinone), GHB (γ-hydroxybutyric acid), αMT (α-methyltryptamine), αET (α-ethyltryptamine), and MDAI (5,6-methylenedioxy-2-aminoindane).

In certain embodiments, the NEI is reboxetine. In certain embodiments, the entactogen is MDMA. In certain embodiments, the subject is administered about 1 mg to about 1000 mg of the NEI.

In certain embodiments, the subject is administered about 1 to about 1000 mg of the entactogen.

In certain embodiments, the NEI is administered to the subject 1 minute to 24 hours before the entactogen is administered to the subject.

In certain embodiments, the NEI and the entactogen are administered concurrently.

In certain embodiments, the NEI and the entactogen are in a single dosage form.

In certain embodiments, the single dosage form includes an anti-abuse formulation.

In certain embodiments, the NEI and the entactogen are independently administered by a route independently selected from the group consisting of oral transdermal, transmucosal, (intra)nasal and (trans)rectal, intravesical, intrapulmonary, intraduodenal, intragastrical, intrathecal, subcutaneous, intramuscular, intradermal, intra-arterial, intravenous, intrabronchial, inhalation, and topical administration.

In certain embodiments, a method of treating, ameliorating, and/or preventing post-traumatic stress disorder in a subject in need thereof is provided. In certain embodiments, the method includes administering to the subject a therapeutically effective amount of at least one α_{2A}-adrenergic receptor agonist (AARA), or a solvate, enantiomer, diastereomer, tautomer, or (pharmaceutically acceptable) salt thereof. In certain embodiments, the method includes administering to the subject a therapeutically effective amount of at least one psychedelic agent, or a solvate, enantiomer, diastereomer, tautomer, or (pharmaceutically acceptable) salt thereof. In certain embodiments, the post-traumatic stress disorder in the subject is treated, ameliorated, or prevented.

In certain embodiments, a method of treating, ameliorating, or preventing post-traumatic stress disorder in a subject in need thereof. In certain embodiments, the method includes administering to the subject a pharmaceutical composition comprising a therapeutically effective amount of a α_{2A}-adrenergic receptor agonist (AARA), or a pharmaceutically acceptable salt, solvate, enantiomer, diastereomer, or tautomer thereof. In certain embodiments, the method includes administering to the subject a pharmaceutical composition comprising a therapeutically effective amount of a psychedelic agent or a pharmaceutically acceptable salt, solvate, enantiomer, diastereomer, or tautomer thereof. In certain embodiments, the pharmaceutical composition comprising the AARA and the pharmaceutical composition comprising the psychedelic agent are formulated as a single dosage form or as separate dosage forms. In certain embodiments, the post-traumatic stress disorder in the subject is treated, ameliorated, or prevented.

In certain embodiments, the AARA, or a solvate, enantiomer, diastereomer, tautomer, or (pharmaceutically acceptable) salt thereof, is selected from the group consisting of clonidine, dexmedetomidine, fadolimidine, guanfacine, guanabenz, guanoxabenz, guanethidine, xylazine, tizanidine, methyldopa, methylnorepinephrine, norepinephrine, detomidine, and lofexidine.

In certain embodiments, the psychedelic agent, or a solvate, enantiomer, diastereomer, tautomer, or (pharmaceutically acceptable) salt thereof, is selected from the group consisting of psilocybin, 2C—B (4-bromo-2,5-dimethoxyphenethylamine), ketamine, DMT (N,N-dimethyltryptamine), 5-MeO-DMT (5-methoxy-dimethyltryptamine), LSA (d-lysergic acid amide), and LSD (lysergic acid diethylamide).

In certain embodiments, the AARA is guanfacine.

In certain embodiments, the psychedelic agent is psilocybin.

In certain embodiments, the subject is administered about 1 to about 1000 mg of the AARA.

In certain embodiments, the subject is administered about 1 to about 1000 mg of the psychedelic agent.

In certain embodiments, the AARA is administered to the subject 1 minute to 24 hours before the psychedelic agent is administered to the subject.

In certain embodiments, the AARA and the psychedelic agent are administered concurrently.

In certain embodiments, the AARA and the psychedelic agent are in a single dosage form.

In certain embodiments, the single dosage form includes an anti-abuse formulation.

In certain embodiments, the AARA and the psychedelic agent are independently administered by a route independently selected from the group consisting of oral transdermal, transmucosal, (intra)nasal and (trans)rectal, intravesical, intrapulmonary, intraduodenal, intragastrical, intrathecal, subcutaneous, intramuscular, intradermal, intra-arterial, intravenous, intrabronchial, inhalation, and topical administration.

The methods described herein include administering to the subject a therapeutically effective amount of at least one compound described herein, which is optionally formulated in a pharmaceutical composition. In various embodiments, a therapeutically effective amount of at least one compound described herein present in a pharmaceutical composition is the only therapeutically active compound in a pharmaceutical composition. In certain embodiments, the method further comprises administering to the subject an additional therapeutic agent that treats PTSD or symptoms thereof.

In certain embodiments, administering the compound(s) described herein to the subject allows for administering a lower dose of the additional therapeutic agent as compared to the dose of the additional therapeutic agent alone that is required to achieve similar results in treating PTSD or symptoms thereof in the subject. For example, in certain embodiments, the compound(s) described herein enhance(s) the activity of the additional therapeutic compound, thereby allowing for a lower dose of the additional therapeutic compound to provide the same effect.

In certain embodiments, the compound(s) described herein and the therapeutic agent are co-administered to the subject. In other embodiments, the compound(s) described herein and the therapeutic agent are coformulated and co-administered to the subject.

In certain embodiments, the subject is a mammal. In other embodiments, the mammal is a human.

### Combination Therapies

The compounds useful within the methods described herein can be used in combination with one or more additional therapeutic agents useful for treating PTSD or symptoms thereof. These additional therapeutic agents may comprise compounds that are commercially available or synthetically accessible to those skilled in the art. These additional therapeutic agents are known to treat or reduce the symptoms, of PTSD or symptoms thereof.

In various embodiments, a synergistic effect is observed when a compound as described herein is administered with one or more additional therapeutic agents or compounds. A synergistic effect may be calculated, for example, using suitable methods such as, for example, the Sigmoid-Eₘₐₓ equation (Holford & Scheiner, 1981, Clin. Pharmacokinet. 6:429-453), the equation of Loewe additivity (Loewe & Muischnek, 1926, Arch. Exp. Pathol Pharmacol. 114:313-326) and the median-effect equation (Chou & Talalay, 1984, Adv. Enzyme Regul. 22:27-55). Each equation referred to above may be applied to experimental data to generate a corresponding graph to aid in assessing the effects of the drug combination. The corresponding graphs associated with the equations referred to above are the concentration-effect curve, isobologram curve and combination index curve, respectively.

### Administration/Dosage/Formulations

The regimen of administration may affect what constitutes an effective amount. The therapeutic formulations may be administered to the subject either prior to or after the onset of PTSD or symptoms thereof. Further, several divided dosages, as well as staggered dosages may be administered daily or sequentially, or the dose may be continuously infused, or may be a bolus injection. Further, the dosages of the therapeutic formulations may be proportionally increased or decreased as indicated by the exigencies of the therapeutic or prophylactic situation.

Administration of the compositions described herein to a patient, preferably a mammal, more preferably a human, may be carried out using known procedures, at dosages and for periods of time effective to treat PTSD or symptoms thereof in the patient. An effective amount of the therapeutic compound necessary to achieve a therapeutic effect may vary according to factors such as the state of the disease or disorder in the patient; the age, sex, and weight of the patient; and the ability of the therapeutic compound to treat a PTSD or symptoms thereof in the patient. Dosage regimens may be adjusted to provide the optimum therapeutic response. For example, several divided doses may be administered daily or the dose may be proportionally reduced as indicated by the exigencies of the therapeutic situation. A non-limiting example of an effective dose range for a therapeutic compound described herein is from about 1 and 5,000 mg/kg of body weight/per day. One of ordinary skill in the art would be able to study the relevant factors and make the determination regarding the effective amount of the therapeutic compound without undue experimentation.

Actual dosage levels of the active ingredients in the pharmaceutical compositions described herein may be varied so as to obtain an amount of the active ingredient that is effective to achieve the desired therapeutic response for a particular patient, composition, and mode of administration, without being toxic to the patient.

In particular, the selected dosage level depends upon a variety of factors including the activity of the particular compound employed, the time of administration, the rate of excretion of the compound, the duration of the treatment, other drugs, compounds or materials used in combination with the compound, the age, sex, weight, condition, general health and prior medical history of the patient being treated, and like factors well, known in the medical arts.

A medical doctor, e.g., physician or veterinarian, having ordinary skill in the art may readily determine and prescribe the effective amount of the pharmaceutical composition required. For example, the physician or veterinarian could start doses of the compounds described herein employed in the pharmaceutical composition at levels lower than that required in order to achieve the desired therapeutic effect and gradually increase the dosage until the desired effect is achieved.

In particular embodiments, it is especially advantageous to formulate the compound in dosage unit form for ease of administration and uniformity of dosage. Dosage unit form as used herein refers to physically discrete units suited as unitary dosages for the patients to be treated; each unit containing a predetermined quantity of therapeutic compound calculated to produce the desired therapeutic effect in association with the required pharmaceutical vehicle. The dosage unit forms of the compound(s) described herein are dictated by and directly dependent on (a) the unique characteristics of the therapeutic compound and the particular therapeutic effect to be achieved, and (b) the limitations inherent in the art of compounding/formulating such a therapeutic compound.

In certain embodiments, the compositions described herein are formulated using one or more pharmaceutically acceptable excipients or carriers. In certain embodiments, the pharmaceutical compositions described herein comprise a therapeutically effective amount of a compound described herein and a pharmaceutically acceptable carrier.

The carrier may be a solvent or dispersion medium containing, for example, water, ethanol, polyol (for example, glycerol, propylene glycol, and liquid polyethylene glycol, and the like), suitable mixtures thereof, and vegetable oils. The proper fluidity may be maintained, for example, by the use of a coating such as lecithin, by the maintenance of the required particle size in the case of dispersion and by the use of surfactants. Prevention of the action of microorganisms may be achieved by various antibacterial and antifungal agents, for example, parabens, chlorobutanol, phenol, ascorbic acid, thimerosal, and the like. In many cases, it is preferable to include isotonic agents, for example, sugars, sodium chloride, or polyalcohols such as mannitol and sorbitol, in the composition. Prolonged absorption of the injectable compositions may be brought about by including in the composition an agent which delays absorption, for example, aluminum monostearate or gelatin.

In certain embodiments, the compositions described herein are administered to the patient in dosages that range from one to five times per day or more. In other embodiments, the compositions described herein are administered to the patient in range of dosages that include, but are not limited to, once every day, every two, days, every three days to once a week, and once every two weeks. It is readily apparent to one skilled in the art that the frequency of administration of the various combination compositions described herein varies from individual to individual depending on many factors including, but not limited to, age, disease or disorder to be treated, gender, overall health, and other factors. Thus, administration of the compounds and compositions described herein should not be construed to be limited to any particular dosage regime and the precise dosage and composition to be administered to any patient is determined by the attending physician taking all other factors about the patient into account.

The compound(s) described herein for administration may be in the range of from about 1 μg to about 10,000 mg, about 20 μg to about 9,500 mg, about 40 μg to about 9,000 mg, about 75 μg to about 8,500 mg, about 150 μg to about 7,500 mg, about 200 μg to about 7,000 mg, about 350 μg to about 6,000 mg, about 500 μg to about 5,000 mg, about 750 μg to about 4,000 mg, about 1 mg to about 3,000 mg, about 10 mg to about 2,500 mg, about 20 mg to about 2,000 mg, about 25 mg to about 1,500 mg, about 30 mg to about 1,000 mg, about 40 mg to about 900 mg, about 50 mg to about 800 mg, about 60 mg to about 750 mg, about 70 mg to about 600 mg, about 80 mg to about 500 mg, and any and all whole or partial increments therebetween.

In some embodiments, the dose of a compound described herein is from about 1 mg and about 2,500 mg. In some embodiments, a dose of a compound described herein used in compositions described herein is less than about 10,000 mg, or less than about 8,000 mg, or less than about 6,000 mg, or less than about 5,000 mg, or less than about 3,000 mg, or less than about 2,000 mg, or less than about 1,000 mg, or less than about 500 mg, or less than about 200 mg, or less than about 50 mg. Similarly, in some embodiments, a dose of a second compound as described herein is less than about 1,000 mg, or less than about 800 mg, or less than about 600 mg, or less than about 500 mg, or less than about 400 mg, or less than about 300 mg, or less than about 200 mg, or less than about 100 mg, or less than about 50 mg, or less than about 40 mg, or less than about 30 mg, or less than about 25 mg, or less than about 20 mg, or less than about 15 mg, or less than about 10 mg, or less than about 5 mg, or less than about 2 mg, or less than about 1 mg, or less than about 0.5 mg, and any and all whole or partial increments thereof.

In certain embodiments, a composition as described herein is a packaged pharmaceutical composition comprising a container holding a therapeutically effective amount of a compound described herein, alone or in combination with a second pharmaceutical agent; and instructions for using the compound to treat, prevent, or reduce one or more symptoms of a disease or disorder in a patient.

Formulations may be employed in admixtures with conventional excipients, i.e., pharmaceutically acceptable organic or inorganic carrier substances suitable for oral, parenteral, nasal, intravenous, subcutaneous, enteral, or any other suitable mode of administration, known to the art. The pharmaceutical preparations may be sterilized and if desired mixed with auxiliary agents, e.g., lubricants, preservatives, stabilizers, wetting agents, emulsifiers, salts for influencing osmotic pressure buffers, coloring, flavoring and/or aromatic substances and the like. They may also be combined where desired with other active agents, e.g., other analgesic agents.

Routes of administration of any of the compositions described herein include oral, nasal, rectal, intravaginal, parenteral, buccal, sublingual or topical. The compounds for use in the compositions described herein can be formulated for administration by any suitable route, such as for oral or parenteral, for example, transdermal, transmucosal (e.g., sublingual, lingual, (trans)buccal, (trans)urethral, vaginal (e.g., trans- and perivaginally), (intra)nasal and (trans)rectal), intravesical, intrapulmonary, intraduodenal, intragastrical, intrathecal, subcutaneous, intramuscular, intradermal, intra-arterial, intravenous, intrabronchial, inhalation, and topical administration.

Suitable compositions and dosage forms include, for example, tablets, capsules, caplets, pills, gel caps, troches, dispersions, suspensions, solutions, syrups, granules, beads, transdermal patches, gels, powders, pellets, magmas, lozenges, creams, pastes, plasters, lotions, discs, suppositories, liquid sprays for nasal or oral administration, dry powder or aerosolized formulations for inhalation, compositions and formulations for intravesical administration and the like. It should be understood that the formulations and compositions described herein are not limited to the particular formulations and compositions that are described herein.

### Oral Administration

For oral application, particularly suitable are tablets, dragees, liquids, drops, suppositories, or capsules, caplets and gelcaps. The compositions intended for oral use may be prepared according to any method known in the art and such compositions may contain one or more agents selected from the group consisting of inert, non-toxic pharmaceutically excipients that are suitable for the manufacture of tablets. Such excipients include, for example an inert diluent such as lactose; granulating and disintegrating agents such as cornstarch; binding agents such as starch; and lubricating agents such as magnesium stearate. The tablets may be uncoated or they may be coated by known techniques for elegance or to delay the release of the active ingredients. Formulations for oral use may also be presented as hard gelatin capsules wherein the active ingredient is mixed with an inert diluent.

For oral administration, the compound(s) described herein can be in the form of tablets or capsules prepared by conventional means with pharmaceutically acceptable excipients such as binding agents (e.g., polyvinylpyrrolidone, hydroxypropylcellulose or hydroxypropyl methylcellulose); fillers (e.g., cornstarch, lactose, microcrystalline cellulose or calcium phosphate); lubricants (e.g., magnesium stearate, talc, or silica); disintegrates (e.g., sodium starch glycollate); or wetting agents (e.g., sodium lauryl sulphate). If desired, the tablets may be coated using suitable methods and coating materials such as OPADRY™ film coating systems available from Colorcon, West Point, Pa. (e.g., OPADRY™ OY Type, OYC Type, Organic Enteric OY-P Type, Aqueous Enteric OY-A Type, OY-PM Type and OPADRY™ White, 32K18400). Liquid preparation for oral administration may be in the form of solutions, syrups or suspensions. The liquid preparations may be prepared by conventional means with pharmaceutically acceptable additives such as suspending agents (e.g., sorbitol syrup, methyl cellulose or hydrogenated edible fats); emulsifying agent (e.g., lecithin or acacia); non-aqueous vehicles (e.g., almond oil, oily esters or ethyl alcohol); and preservatives (e.g., methyl or propyl p-hydroxy benzoates or sorbic acid).

Compositions as described herein can be prepared, packaged, or sold in a formulation suitable for oral or buccal administration. A tablet that includes a compound as described herein can, for example, be made by compressing or molding the active ingredient, optionally with one or more additional ingredients. Compressed tablets may be prepared by compressing, in a suitable device, the active ingredient in a free-flowing form such as a powder or granular preparation, optionally mixed with one or more of a binder, a lubricant, an excipient, a surface active agent, and a dispersing agent. Molded tablets may be made by molding, in a suitable device, a mixture of the active ingredient, a pharmaceutically acceptable carrier, and at least sufficient liquid to moisten the mixture. Pharmaceutically acceptable excipients used in the manufacture of tablets include, but are not limited to, inert diluents, granulating and disintegrating agents, dispersing agents, surface-active agents, disintegrating agents, binding agents, and lubricating agents.

Suitable dispersing agents include, but are not limited to, potato starch, sodium starch glycollate, poloxamer 407, or poloxamer 188. One or more dispersing agents can each be individually present in the composition in an amount of about 0.01% w/w to about 90% w/w relative to weight of the dosage form. One or more dispersing agents can each be individually present in the composition in an amount of at least, greater than, or less than about 0.01%, 0.05%, 0.1%, 0.5%, 1%, 2%, 3%, 4%, 5%, 10%, 15%, 20%, 25%, 30%, 35%, 40%, 45%, 50%, 55%, 60%, 65%, 70%, 75%, 80%, 85%, or 90% w/w relative to weight of the dosage form.

Surface-active agents (surfactants) include cationic, anionic, or non-ionic surfactants, or combinations thereof. Suitable surfactants include, but are not limited to, behentrimonium chloride, benzalkonium chloride, benzethonium chloride, benzododecinium bromide, carbethopendecinium bromide, cetalkonium chloride, cetrimonium bromide, cetrimonium chloride, cetylpyridine chloride, didecyldimethylammonium chloride, dimethyldioctadecylammonium bromide, dimethyldioctadecylammonium chloride, domiphen bromide, lauryl methyl gluceth-10 hydroxypropyl dimonium chloride, tetramethylammonium hydroxide, thonzonium bromide, stearalkonium chloride, octenidine dihydrochloride, olaflur, N-oleyl-1,3-propanediamine, 2-acrylamido-2-methylpropane sulfonic acid, alkylbenzene sulfonates, ammonium lauryl sulfate, ammonium perfluorononanoate, docusate, disodium cocoamphodiacetate, magnesium laureth sulfate, perfluorobutanesulfonic acid, perfluorononanoic acid, perfluorooctanesulfonic acid, perfluorooctanoic acid, potassium lauryl sulfate, sodium alkyl sulfate, sodium dodecyl sulfate, sodium laurate, sodium laureth sulfate, sodium lauroyl sarcosinate, sodium myreth sulfate, sodium nonanoyloxybenzenesulfonate, sodium pareth sulfate, sodium stearate, sodium sulfosuccinate esters, cetomacrogol 1000, cetostearyl alcohol, cetyl alcohol, cocamide diethanolamine, cocamide monoethanolamine, decyl glucoside, decyl polyglucose, glycerol monostearate, octylphenoxypolyethoxyethanol CA-630, isoceteth-20, lauryl glucoside, octylphenoxypolyethoxyethanol P-40, Nonoxynol-9, Nonoxynols, nonyl phenoxypolyethoxylethanol (NP-40), octaethylene glycol monododecyl ether, N-octyl beta-D-thioglucopyranoside, octyl glucoside, oleyl alcohol, PEG-10 sunflower glycerides, pentaethylene glycol monododecyl ether, polidocanol, poloxamer, poloxamer 407, polyethoxylated tallow amine, polyglycerol polyricinoleate, polysorbate, polysorbate 20, polysorbate 80, sorbitan, sorbitan monolaurate, sorbitan monostearate, sorbitan tristearate, stearyl alcohol, surfactin, Triton X-100, and Tween 80. One or more surfactants can each be individually present in the composition in an amount of about 0.01% w/w to about 90% w/w relative to weight of the dosage form. One or more surfactants can each be individually present in the composition in an amount of at least, greater than, or less than about 0.01%, 0.05%, 0.1%, 0.5%, 1%, 2%, 3%, 4%, 5%, 10%, 15%, 20%, 25%, 30%, 35%, 40%, 45%, 50%, 55%, 60%, 65%, 70%, 75%, 80%, 85%, or 90% w/w relative to weight of the dosage form.

Suitable diluents include, but are not limited to, calcium carbonate, magnesium carbonate, magnesium oxide, sodium carbonate, lactose, microcrystalline cellulose, calcium phosphate, calcium hydrogen phosphate, and sodium phosphate, Cellactose® 80 (75% α-lactose monohydrate and 25% cellulose powder), mannitol, pre-gelatinized starch, starch, sucrose, sodium chloride, talc, anhydrous lactose, and granulated lactose. One or more diluents can each be individually present in the composition in an amount of about 0.01% w/w to about 90% w/w relative to weight of the dosage form. One or more diluents can each be individually present in the composition in an amount of at least, greater than, or less than about 0.01%, 0.05%, 0.1%, 0.5%1, 1%, 2%, 3%, 4%, 5%, 10%, 15%, 20%, 25%, 30%, 35%, 40%, 45%, 50%, 55%, 60%, 65%, 70%, 75%, 80%, 85%, or 90% w/w relative to weight of the dosage form.

Suitable granulating and disintegrating agents include, but are not limited to, sucrose, copovidone, corn starch, microcrystalline cellulose, methyl cellulose, sodium starch glycollate, pregelatinized starch, povidone, sodium carboxy methyl cellulose, sodium alginate, citric acid, croscarmellose sodium, cellulose, carboxymethylcellulose calcium, colloidal silicone dioxide, crosspovidone and alginic acid. One or more granulating or disintegrating agents can each be individually present in the composition in an amount of about 0.01% w/w to about 90% w/w relative to weight of the dosage form. One or more granulating or disintegrating agents can each be individually present in the composition in an amount of at least, greater than, or less than about 0.01%, 0.05%, 0.1%, 0.5%, 1%, 2%, 3%, 4%, 5%0, 10%, 15%, 20%, 25%, 30%, 35%, 40%, 45%, 50%, 55%, 60%, 65%, 70%, 75%, 80%, 85%, or 90% w/w relative to weight of the dosage form.

Suitable binding agents include, but are not limited to, gelatin, acacia, pre-gelatinized maize starch, polyvinylpyrrolidone, anhydrous lactose, lactose monohydrate, hydroxypropyl methylcellulose, methylcellulose, povidone, polyacrylamides, sucrose, dextrose, maltose, gelatin, polyethylene glycol. One or more binding agents can each be individually present in the composition in an amount of about 0.01% w/w to about 90% w/w relative to weight of the dosage form. One or more binding agents can each be individually present in the composition in an amount of at least, greater than, or less than about 0.01%, 0.05%, 0.1%, 0.5%, 1%, 2%, 3%, 4%, 5%, 10%, 15%, 20%, 25%, 30%, 35%, 40%, 45%, 50%, 55%, 60%, 65%, 70%, 75%, 80%, 85%, or 90% w/w relative to weight of the dosage form.

Suitable lubricating agents include, but are not limited to, magnesium stearate, calcium stearate, hydrogenated castor oil, glyceryl monostearate, glyceryl behenate, mineral oil, polyethylene glycol, poloxamer 407, poloxamer 188, sodium laureth sulfate, sodium benzoate, stearic acid, sodium stearyl fumarate, silica, and talc. One or more lubricating agents can each be individually present in the composition in an amount of about 0.01% w/w to about 90% w/w relative to weight of the dosage form. One or more lubricating agents can each be individually present in the composition in an amount of at least, greater than, or less than about 0.01%, 0.05%, 0.1%, 0.5%1, 1%, 2%, 3%, 4%, 5%, 10%, 15%, 20%, 25%, 30%, 35%, 40%, 45%, 50%, 55%, 60%, 65%, 70%, 75%, 80%, 85%, or 90% w/w relative to weight of the dosage form.

Tablets can be non-coated or they may be coated using known methods to achieve delayed disintegration in the gastrointestinal tract of a subject, thereby providing sustained release and absorption of the active ingredient. By way of example, a material such as glyceryl monostearate or glyceryl distearate may be used to coat tablets. Further by way of example, tablets may be coated using methods described in U.S. Pat. Nos. 4,256,108; 4,160,452; and U.S. Pat. No. 4,265,874 to form osmotically controlled release tablets. Tablets may further comprise a sweetening agent, a flavoring agent, a coloring agent, a preservative, or some combination of these in order to provide for pharmaceutically elegant and palatable preparation.

Tablets can also be enterically coated such that the coating begins to dissolve at a certain pH, such as at about pH 5.0 to about pH 7.5, thereby releasing a compound as described herein. The coating can contain, for example, EUDRAGIT® L, S, FS, and/or E polymers with acidic or alkaline groups to allow release of a compound as described herein in a particular location, including in any desired section(s) of the intestine. The coating can also contain, for example, EUDRAGIT® RL and/or RS polymers with cationic or neutral groups to allow for time controlled release of a compound as described herein by pH-independent swelling.

### Parenteral Administration

For parenteral administration, the compounds as described herein may be formulated for injection or infusion, for example, intravenous, intramuscular or subcutaneous injection or infusion, or for administration in a bolus dose and/or continuous infusion. Suspensions, solutions or emulsions in an oily or aqueous vehicle, optionally containing other formulatory agents such as suspending, stabilizing and/or dispersing agents may be used.

Sterile injectable forms of the compositions described herein may be aqueous or oleaginous suspension. These suspensions may be formulated according to techniques known in the art using suitable dispersing or wetting agents and suspending agents. The sterile injectable preparation may also be a sterile injectable solution or suspension in a non-toxic parenterally-acceptable diluent or solvent, for example as a solution in 1, 3-butanediol. Among the acceptable vehicles and solvents that may be employed are water, Ringer's solution and isotonic sodium chloride solution. Sterile, fixed oils are conventionally employed as a solvent or suspending medium. For this purpose, any bland fixed oil may be employed including synthetic mono- or di-glycerides. Fatty acids, such as oleic acid and its glyceride derivatives are useful in the preparation of injectables, as are natural pharmaceutically acceptable oils, such as olive oil or castor oil, especially in their polyoxyethylated versions. These oil solutions or suspensions may also contain a long-chain alcohol diluent or dispersant, such as such as lauryl, stearyl, or oleyl alcohols, or similar alcohol.

### Additional Administration Forms

Additional dosage forms suitable for use with the compound(s) and compositions described herein include dosage forms as described in U.S. Pat. Nos. 6,340,475; 6,488,962; 6,451,808; 5,972,389; 5,582,837; and 5,007,790. Additional dosage forms suitable for use with the compound(s) and compositions described herein also include dosage forms as described in U.S. Patent Applications Nos. 20030147952; 20030104062; 20030104053; 20030044466; 20030039688; and 20020051820. Additional dosage forms suitable for use with the compound(s) and compositions described herein also include dosage forms as described in PCT Applications Nos. WO 03/35041; WO 03/35040; WO 03/35029; WO 03/35177; WO 03/35039; WO 02/96404; WO 02/32416; WO 01/97783; WO 01/56544; WO 01/32217; WO 98/55107; WO 98/11879; WO 97/47285; WO 93/18755; and WO 90/11757.

### Controlled Release Formulations and Drug Delivery Systems

In certain embodiments, the formulations described herein can be, but are not limited to, short-term, rapid-offset, as well as controlled, for example, sustained release, delayed release and pulsatile release formulations.

The term sustained release is used in its conventional sense to refer to a drug formulation that provides for gradual release of a drug over an extended period of time, and that may, although not necessarily, result in substantially constant blood levels of a drug over an extended time period. The period of time may be as long as a month or more and should be a release which is longer that the same amount of agent administered in bolus form.

For sustained release, the compounds may be formulated with a suitable polymer or hydrophobic material which provides sustained release properties to the compounds. As such, the compounds for use with the method(s) described herein may be administered in the form of microparticles, for example, by injection or in the form of wafers or discs by implantation.

In some cases, the dosage forms to be used can be provided as slow or controlled-release of one or more active ingredients therein using, for example, hydropropylmethyl cellulose, other polymer matrices, gels, permeable membranes, osmotic systems, multilayer coatings, microparticles, liposomes, or microspheres or a combination thereof to provide the desired release profile in varying proportions. Suitable controlled-release formulations known to those of ordinary skill in the art, including those described herein, can be readily selected for use with the pharmaceutical compositions described herein. Thus, single unit dosage forms suitable for oral administration, such as tablets, capsules, gelcaps, and caplets, that are adapted for controlled-release are encompassed by the compositions and dosage forms described herein.

Most controlled-release pharmaceutical products have a common goal of improving drug therapy over that achieved by their non-controlled counterparts. Ideally, the use of an optimally designed controlled-release preparation in medical treatment is characterized by a minimum of drug substance being employed to cure or control the condition in a minimum amount of time. Advantages of controlled-release formulations include extended activity of the drug, reduced dosage frequency, and increased patient compliance. In addition, controlled-release formulations can be used to affect the time of onset of action or other characteristics, such as blood level of the drug, and thus can affect the occurrence of side effects.

Most controlled-release formulations are designed to initially release an amount of drug that promptly produces the desired therapeutic effect, and gradually and continually release of other amounts of drug to maintain this level of therapeutic effect over an extended period of time. In order to maintain this constant level of drug in the body, the drug must be released from the dosage form at a rate that will replace the amount of drug being metabolized and excreted from the body.

Controlled-release of an active ingredient can be stimulated by various inducers, for example pH, temperature, enzymes, water, or other physiological conditions or compounds. The term “controlled-release component” is defined herein as a compound or compounds, including, but not limited to, polymers, polymer matrices, gels, permeable membranes, liposomes, or microspheres or a combination thereof that facilitates the controlled-release of the active ingredient. In one embodiment, the compound(s) described herein are administered to a patient, alone or in combination with another pharmaceutical agent, using a sustained release formulation. In one embodiment, the compound(s) described herein are administered to a patient, alone or in combination with another pharmaceutical agent, using a sustained release formulation.

The term delayed release is used herein in its conventional sense to refer to a drug formulation that provides for an initial release of the drug after some delay following drug administration and that mat, although not necessarily, includes a delay of from about 10 minutes up to about 12 hours.

The term pulsatile release is used herein in its conventional sense to refer to a drug formulation that provides release of the drug in such a way as to produce pulsed plasma profiles of the drug after drug administration.

The term immediate release is used in its conventional sense to refer to a drug formulation that provides for release of the drug immediately after drug administration.

As used herein, short-term refers to any period of time up to and including about 8 hours, about 7 hours, about 6 hours, about 5 hours, about 4 hours, about 3 hours, about 2 hours, about 1 hour, about 40 minutes, about 20 minutes, or about 10 minutes and any or all whole or partial increments thereof after drug administration after drug administration.

As used herein, rapid-offset refers to any period of time up to and including about 8 hours, about 7 hours, about 6 hours, about 5 hours, about 4 hours, about 3 hours, about 2 hours, about 1 hour, about 40 minutes, about 20 minutes, or about 10 minutes, and any and all whole or partial increments thereof after drug administration.

### Dosing

The therapeutically effective amount or dose of a compound described herein depends on the age, sex and weight of the patient, the current medical condition of the patient and the progression of PTSD or symptoms thereof in the patient being treated. The skilled artisan is able to determine appropriate dosages depending on these and other factors.

A suitable dose of a compound described herein can be in the range of from about 0.01 mg to about 5,000 mg per day, such as from about 0.1 mg to about 1,000 mg, for example, from about 1 mg to about 500 mg, such as about 5 mg to about 250 mg per day. The dose may be administered in a single dosage or in multiple dosages, for example from 1 to 4 or more times per day. When multiple dosages are used, the amount of each dosage may be the same or different. For example, a dose of 1 mg per day may be administered as two 0.5 mg doses, with about a 12-hour interval between doses.

It is understood that the amount of compound dosed per day may be administered, in non-limiting examples, every day, every other day, every 2 days, every 3 days, every 4 days, or every 5 days. For example, with every other day administration, a 5 mg per day dose may be initiated on Monday with a first subsequent 5 mg per day dose administered on Wednesday, a second subsequent 5 mg per day dose administered on Friday, and so on.

In the case wherein the patient's status does improve, upon the doctor's discretion the administration of the compound(s) described herein is optionally given continuously; alternatively, the dose of drug being administered is temporarily reduced or temporarily suspended for a certain length of time (i.e., a “drug holiday”). The length of the drug holiday optionally varies between 2 days and 1 year, including by way of example only, 2 days, 3 days, 4 days, 5 days, 6 days, 7 days, 10 days, 12 days, 15 days, 20 days, 28 days, 35 days, 50 days, 70 days, 100 days, 120 days, 150 days, 180 days, 200 days, 250 days, 280 days, 300 days, 320 days, 350 days, or 365 days. The dose reduction during a drug holiday includes from 10%-100%, including, by way of example only, 10%, 15%, 20%, 25%, 30%, 35%, 40%, 45%, 50%, 55%, 60%, 65%, 70%, 75%, 80%, 85%, 90%, 95%, or 100%.

Once improvement of the patient's conditions has occurred, a maintenance dose is administered if necessary. Subsequently, the dosage or the frequency of administration, or both, is reduced to a level at which the improved disease is retained. In certain embodiments, patients require intermittent treatment on a long-term basis upon any recurrence of symptoms and/or infection.

The compounds described herein can be formulated in unit dosage form. The term “unit dosage form” refers to physically discrete units suitable as unitary dosage for patients undergoing treatment, with each unit containing a predetermined quantity of active material calculated to produce the desired therapeutic effect, optionally in association with a suitable pharmaceutical carrier. The unit dosage form may be for a single daily dose or one of multiple daily doses (e.g., about 1 to 4 or more times per day). When multiple daily doses are used, the unit dosage form may be the same or different for each dose.

Toxicity and therapeutic efficacy of such therapeutic regimens are optionally determined in cell cultures or experimental animals, including, but not limited to, the determination of the LD₅₀ (the dose lethal to 50% of the population) and the ED₅₀ (the dose therapeutically effective in 50% of the population). The dose ratio between the toxic and therapeutic effects is the therapeutic index, which is expressed as the ratio between LD₅₀ and ED₅₀. The data obtained from cell culture assays and animal studies are optionally used in formulating a range of dosage for use in human. The dosage of such compounds lies preferably within a range of circulating concentrations that include the ED₅₀ with minimal toxicity. The dosage optionally varies within this range depending upon the dosage form employed and the route of administration utilized.

### Examples

Various embodiments of the present application can be better understood by reference to the following Examples which are offered by way of illustration. The scope of the present application is not limited to the Examples given herein.

### Experimental Methods

### Animals

All experiments used wild-type C57BL6/J background mice purchased from Jackson laboratories at 6-8 weeks of age. Animals were group housed, kept on a 12 h/12 h light-dark cycle, and were provided ad libitum chow and water. All experiments were performed during the light cycle (7:00-19:00).

### Drug Preparation and Administration

Drugs were administered intraperitoneal (IP) at 0.01 mL/kg using insulin syringes. MDMA, psilocybin, reboxetine, and guanfacine were purchased from Cayman Chemicals (Ann Arbor, MI) and dissolved in saline. Yohimbine was purchased from Sigma Aldrich (St. Louis, MO) and dissolved in distilled water.

For fear conditioning studies, drug was administered 30 minutes before the start of the trail. In 5-HT and NE photometry studies, drug was administered immediately before placing the mouse in an empty box, 5 minutes after the start of recording to get a baseline measure. For dose response curves of HTR, drug was administered immediately before the start of the trial. In the HTR and photometry that involved reboxetine and fluoxetine, these inhibitors were injected one minute before entactogen administration. Guanfacine was injected 15 minutes before reboxetine or psilocybin administration. Yohimbine was administered 2 minutes before MDMA administration in HTR studies.

### Surgeries

For all stereotaxic surgical procedures, mice were first anesthetized with isoflurane in oxygen (3-5% during induction, slowly lowered throughout the surgery to 1-2%) while placed in a stereotaxic apparatus (Stoelting). Eyes were lubricated with ophthalmic ointment. Hair was removed with scissors or an electric razor (Phillips), and an incision in the scalp was made to expose the skull. A craniotomy was then made with either a rotary tool (Dremel) or a dental drill above the mPFC. 0.5 μL of AAV9-hSyn-NE2h (WZ Biosciences) virus (1×10¹³ genome copies per mL) was then loaded into a Hamilton syringe. The tip of the syringe was then placed over the craniotomy and then lowered to the injection site. Injections were targeted to the prefrontal cortex (AP: +1.6-2.1 mm AP, ML: ±0.3 mm, DV: 1.3 mm below dura). Virus was then injected at a rate of 0.1 μL/min. After injection, the syringe was left in place for at least 5 minutes, then slowly raised out of the craniotomy. Fiber implantation was done directly after virus injection. Fiber optic implants (0.2 mm core, Neurophotometrics) were cut to 3 mm length. Implants were held in the stereotaxic apparatus and lowered to the viral injection coordinates. Implants were then secured with quick adhesive cement (C&B Metabond, Parkell). Mice were then removed from the apparatus and recovered in the home cage. All mice received carprofen (5 mg/kg, Zoetis) intraperitoneally after surgery and for two days following surgery. Any behavioral testing took place at least 3 weeks after surgery.

### Fiber Photometry

Mice used in fiber photometry experiments were subjected to the surgical procedures described herein approximately 4-8 weeks prior to experimental sessions. A crossover design was used for these experiments, in which each animal received all drug conditions over the course of several days (one day between each condition). The cohort of animals used to measure 5-HT received MDMA (12 mg/kg) and saline. The cohort for NE received MDMA (12 mg/kg) and saline. The NE blocker photometry studies with fluoxetine and reboxetine were done with the cohort from the NE study, with all animals receiving fluoxetine (10 mg/kg)+MDMA (12 mg/kg) and reboxetine (10 mg/kg)+MDMA (12 mg/kg) over two experimental sessions with one day between them. In this case, when animals were used in two photometry studies, the cohort was run at least 2 weeks after the end of the first experiment.

Fiber photometry of sensor fluorescence was performed using an FP3002 fiber photometry system (Neurophotometrics). A fiber optic cable (Doric) connected to the FP3002 was attached to fiber optic implants via a ceramic sleeve. Light at the excitation wavelength (470 nm) and an isosbestic control wavelength (415 nm) was produced from LEDs and propagated through the fiber optic cable to the fiber optic implant. Emission light from the fluorescent sensor was then captured using an sCMOS camera. Excitation wavelengths were used at 10% power, and 470 nm and 415 nm excitation was interleaved at a total sampling rate of 40 Hz, producing 20 Hz recordings for each channel. Only analysis using the 470 nm excitation channel was performed, since 415 nm is not a true isosbestic point for GRABNE.

Recordings were saved and analyzed offline. For each recording, the injection fluorescence change was calculated as a percent change from the pre-injection average. Fluorescence from the 470 nm channel was extracted from 120 s-240 s and used as a baseline. The percent change for each trial was calculated as a percent change from the average signal from 120 s to 240 s after the beginning of the recording before drug administration: that is, dF/F=((signal)−(average signal from 120 s to 240 s after onset of recording))/(average signal from 120 s to 240 s after onset recording). Analyses were performed using custom MATLAB scripts, and statistical analysis were performed using MATLAB or GraphPad Prism 9.

### Head Twitch Response

Head twitch response (HTR) was evaluated using C57BL/6J mice (ages 7-12 weeks). Mice were ear tagged >3 days after arrival to the animal facility and allowed to rest for at least 3 days before behavioral testing. Small magnets (SuperMagnetMan—N45 magnet 3 mm diameter, 0.5 mm thickness) were glued to ear tags (Stoelting—La Pias Aluminum Ear Tags)>2 days before mice were ear tagged. The experimental setup was adapted from Gonzales-Maeso and Kwan, including experimental apparatus and Matlab code for analysis.

Doses were randomly assigned, and age matched controls were injected with saline and randomly interspersed with drug conditions. At least three animals per sex were used for each drug dose and condition. In the event that animals were used in more than one HTR experiment, experimental sessions took place at least one week apart. Two animals were run at a time. Immediately after i.p. injection of psychedelic or entactogen drug, animals were placed in separate plastic boxes within a larger chamber (Home Depot—28 in. W×32 in. H×21.5 in. D), and HTR was measured for 10 minutes for dose response curves, and 30 minutes for other circuit level HTR experiments (FIGS. 2A-2C, 3A-3C, and 4A-4C). A small lamp was placed in the back of the larger chamber, and a high-speed camera (Basler—acA1920-155 um) was suspended from the ceiling to record video of the animals in the plastic boxes. Between each recording, the plastic boxes were cleaned with 70% ethanol. Head twitch counts were imported into Prism and used to make graphs and run appropriate statistics for analysis.

The terms and expressions employed herein are used as terms of description and not of limitation, and there is no intention in the use of such terms and expressions of excluding any equivalents of the features shown and described or portions thereof, but it is recognized that various modifications are possible within the scope of the embodiments of the present application. Thus, it should be understood that although the present application describes specific embodiments and optional features, modification and variation of the compositions, methods, and concepts herein disclosed may be resorted to by those of ordinary skill in the art, and that such modifications and variations are considered to be within the scope of embodiments of the present application.

### Enumerated Embodiments

The following enumerated embodiments are provided, the numbering of which is not to be construed as designating levels of importance:

Embodiment 1 provides a method of treating, ameliorating, and/or preventing post-traumatic stress disorder in a subject in need thereof, the method comprising:
administering to the subject:
i) a pharmaceutical composition comprising a therapeutically effective amount of a norepinephrine inhibitor (NEI), or a pharmaceutically acceptable salt, solvate, enantiomer, diastereomer, or tautomer thereof, and
ii) a pharmaceutical composition comprising a therapeutically effective amount of an entactogen, or a pharmaceutically acceptable salt, solvate, enantiomer, diastereomer, or tautomer thereof;
wherein the pharmaceutical composition comprising the NEI and the pharmaceutical composition comprising the entactogen are formulated as a single dosage form or as separate dosage forms; and
wherein the post-traumatic stress disorder in the subject is treated, ameliorated, or prevented.

Embodiment 2 provides the method of embodiment 1, wherein the NEI is selected from the group consisting of atomoxetine ((R)—N-methyl-3-phenyl-3-(o-tolyloxy)propan-1-amine), reboxetine (rel-(2R)-2-[(R)-(2-ethoxyphenoxy)(phenyl)methyl]morpholine), viloxazine ((RS)-2-[(2-ethoxyphenoxy)methyl]morpholine), amedalin (UK-3540-1), daledalin (UK-3557-15), edivoxetine (LY-2216684), esreboxetine (AXS-14; PNU-165442G), lortalamine (LM-1404), nisoxetine (LY-94,939), talopram (tasulopram) (Lu 3-010), talsupram (Lu 5-005), and tandamine (AY-23,946), or a pharmaceutically acceptable salt, solvate, enantiomer, diastereomer, or tautomer thereof.

Embodiment 3 provides the method of embodiment 1, wherein the entactogen is selected from the group consisting of MDMA (3,4-methylenedioxymethamphetamine), MDA (3,4-methylenedioxyamphetamine), MDEA (3,4-methylenedioxy-N-ethylamphetamine), MDOH (3,4-methylenedioxy-N-hydroxyamphetamine), MBDB (1,3-benzodioxolyl-N-methylbutanamine), 5-APB (1-benzofuran-5-ylpropan-2-amine), 5-MAPB (1-(benzofuran-5-yl)-N-methylpropan-2-amine), 6-APB (6-(2-aminopropyl)benzofuran), 6-MAPB (1-(benzofuran-6-yl)-N-methylpropan-2-amine), methylone (3,4-methylenedioxy-N-methylcathinone), mephedrone (4-methylmethcathinone), GHB (γ-hydroxybutyric acid), αMT (α-methyltryptamine), αET (α-ethyltryptamine), and MDAI (5,6-methylenedioxy-2-aminoindane), or a pharmaceutically acceptable salt, solvate, enantiomer, diastereomer, or tautomer thereof.

Embodiment 4 provides the method of any one of embodiments 1-3, wherein the NEI is reboxetine.

Embodiment 5 provides the method of any one of embodiments 1-4, wherein the entactogen is MDMA.

Embodiment 6 provides the method of any one of embodiments 1-5, wherein the subject is administered about 1 to about 1000 mg of the NEI.

Embodiment 7 provides the method of any one of embodiments 1-6, wherein the subject is administered about 1 to about 1000 mg of the entactogen.

Embodiment 8 provides the method of any one of embodiments 1-7, wherein the pharmaceutical composition comprising the NEI and the pharmaceutical composition comprising the entactogen are formulated as separate dosage forms.

Embodiment 9 provides the method of any one of embodiments 1-8, wherein the pharmaceutical composition comprising the NEI is administered to the subject about 1 minute to about 24 hours before the pharmaceutical composition comprising the entactogen is administered to the subject.

Embodiment 10 provides the method of any one of embodiments 1-9, wherein the pharmaceutical composition comprising the NEI and the pharmaceutical composition comprising the entactogen are administered concurrently.

Embodiment 11 provides the method of any one of embodiments 1-10, wherein the pharmaceutical composition comprising the NEI and the pharmaceutical composition comprising the entactogen are formulated as a single dosage form.

Embodiment 12 provides the method of any one of embodiments 1-11, wherein the single dosage form comprises an anti-abuse formulation.

Embodiment 13 provides the method of any one of embodiments 1-12, wherein the pharmaceutical composition comprising the NEI and the pharmaceutical composition comprising the entactogen are independently administered by a route independently selected from the group consisting of oral transdermal, transmucosal, (intra)nasal and (trans)rectal, intravesical, intrapulmonary, intraduodenal, intragastrical, intrathecal, subcutaneous, intramuscular, intradermal, intra-arterial, intravenous, intrabronchial, inhalation, and topical administration.

Embodiment 14 provides the method of any one of embodiments 1-14, wherein the pharmaceutical composition comprising the NEI and the pharmaceutical composition comprising the entactogen each independently further comprise at least one pharmaceutically acceptable carrier or excipient.

Embodiment 15 provides a method of treating, ameliorating, or preventing post-traumatic stress disorder in a subject in need thereof, the method comprising: administering to the subject:
i) a pharmaceutical composition comprising a therapeutically effective amount of a α_{2A}-adrenergic receptor agonist (AARA), or a pharmaceutically acceptable salt, solvate, enantiomer, diastereomer, or tautomer thereof; and
ii) a pharmaceutical composition comprising a therapeutically effective amount of a psychedelic agent or a pharmaceutically acceptable salt, solvate, enantiomer, diastereomer, or tautomer thereof;
wherein the pharmaceutical composition comprising the AARA and the pharmaceutical composition comprising the psychedelic agent are formulated as a single dosage form or as separate dosage forms; and
wherein the post-traumatic stress disorder in the subject is treated, ameliorated, or prevented.

Embodiment 16 provides the method of any embodiment 15, wherein the AARA is selected from the group consisting of clonidine, dexmedetomidine, fadolimidine, guanfacine, guanabenz, guanoxabenz, guanethidine, xylazine, tizanidine, methyldopa, methylnorepinephrine, norepinephrine, detomidine, and lofexidine, or a pharmaceutically acceptable salt, solvate, enantiomer, diastereomer, or tautomer thereof.

Embodiment 17 provides the method of any one of embodiments 15-16, wherein the psychedelic agent is selected from the group consisting of psilocybin, 2C—B (4-bromo-2,5-dimethoxyphenethylamine), ketamine, DMT (N,N-dimethyltryptamine), 5-MeO-DMT (5-methoxy-dimethyltryptamine), LSA (d-lysergic acid amide), and LSD (lysergic acid diethylamide) or a pharmaceutically acceptable salt, solvate, enantiomer, diastereomer, or tautomer thereof.

Embodiment 18 provides the method of any one of embodiments 15-17, wherein the AARA is guanfacine.

Embodiment 19 provides the method of any one of embodiments 15-18, wherein the psychedelic agent is psilocybin.

Embodiment 20 provides the method of any one of embodiments 15-19, wherein the subject is administered about 1 to about 1000 mg of the AARA.

Embodiment 21 provides the method of any one of embodiments 15-20, wherein the subject is administered about 1 to about 1000 mg of the psychedelic agent.

Embodiment 22 provides the method of any one of embodiments 15-21, wherein the pharmaceutical composition comprising the AARA and the pharmaceutical composition comprising the psychedelic agent are formulated as separate dosage forms.

Embodiment 23 provides the method of any one of embodiments 15-22, wherein the pharmaceutical composition comprising the AARA is administered to the subject about 1 minute to about 24 hours before the pharmaceutical composition comprising the psychedelic agent is administered to the subject.

Embodiment 24 provides the method of any one of embodiments 15-23, wherein the pharmaceutical composition comprising the AARA and the pharmaceutical composition comprising the psychedelic agent are administered concurrently.

Embodiment 25 provides the method of any one of embodiments 15-24, wherein the pharmaceutical composition comprising the AARA and the pharmaceutical composition comprising the psychedelic agent are formulated as a single dosage form.

Embodiment 26 provides the method of any one of embodiments 15-25, wherein the single dosage form comprises an anti-abuse formulation.

Embodiment 27 provides the method of any one of embodiments 15-26, wherein the pharmaceutical composition comprising the AARA and the pharmaceutical composition comprising the psychedelic agent are independently administered by a route independently selected from the group consisting of oral transdermal, transmucosal, (intra)nasal and (trans)rectal, intravesical, intrapulmonary, intraduodenal, intragastrical, intrathecal, subcutaneous, intramuscular, intradermal, intra-arterial, intravenous, intrabronchial, inhalation, and topical administration.

Embodiment 28 provides the method of any one of embodiments 15-27, wherein the pharmaceutical composition comprising the NEI and the pharmaceutical composition comprising the entactogen each independently further comprise at least one pharmaceutically acceptable carrier or excipient.

1. A method of treating or ameliorating post-traumatic stress disorder in a subject in need thereof, the method comprising:
administering to the subject:
i) a pharmaceutical composition comprising a therapeutically effective amount of a norepinephrine inhibitor (NEI), or a pharmaceutically acceptable salt, solvate, enantiomer, diastereomer, or tautomer thereof, and
ii) a pharmaceutical composition comprising a therapeutically effective amount of an entactogen, or a pharmaceutically acceptable salt, solvate, enantiomer, diastereomer, or tautomer thereof,
wherein the pharmaceutical composition comprising the NEI and the pharmaceutical composition comprising the entactogen are formulated as a single dosage form or as separate dosage forms; and
wherein the post-traumatic stress disorder in the subject is treated or ameliorated.

2. The method of claim 1, wherein the NEI is selected from the group consisting of atomoxetine ((R)—N-methyl-3-phenyl-3-(o-tolyloxy)propan-1-amine), reboxetine (rel-(2R)-2-[(R)-(2-ethoxyphenoxy)(phenyl)methyl]morpholine), viloxazine ((RS)-2-[(2-ethoxyphenoxy)methyl]morpholine), amedalin (UK-3540-1), daledalin (UK-3557-15), edivoxetine (LY-2216684), esreboxetine (AXS-14; PNU-165442G), lortalamine (LM-1404), nisoxetine (LY-94,939), talopram (tasulopram) (Lu 3-010), talsupram (Lu 5-005), and tandamine (AY-23,946), or a pharmaceutically acceptable salt, solvate, enantiomer, diastereomer, or tautomer thereof.

3. The method of claim 1, wherein the entactogen is selected from the group consisting of MDMA (3,4-methylenedioxymethamphetamine), MDA (3,4-methylenedioxyamphetamine), MDEA (3,4-methylenedioxy-N-ethylamphetamine), MDOH (3,4-methylenedioxy-N-hydroxyamphetamine), MBDB (1,3-benzodioxolyl-N-methylbutanamine), 5-APB (1-benzofuran-5-ylpropan-2-amine), 5-MAPB (1-(benzofuran-5-yl)-N-methylpropan-2-amine), 6-APB (6-(2-aminopropyl)benzofuran), 6-MAPB (1-(benzofuran-6-yl)-N-methylpropan-2-amine), methylone (3,4-methylenedioxy-N-methylcathinone), mephedrone (4-methylmetheathinone), GHB (γ-hydroxybutyric acid), αMT (α-methyltryptamine), αET (α-ethyltryptamine), and MDAI (5,6-methylenedioxy-2-aminoindane), or a pharmaceutically acceptable salt, solvate, enantiomer, diastereomer, or tautomer thereof.

4. The method of claim 2, wherein the NEI is reboxetine.

5. The method of claim 3, wherein the entactogen is MDMA.

6. The method of claim 1, wherein at least one of the following applies:
i) the subject is administered about 1 to about 1000 mg of the NEI,
ii) the subject is administered about 1 to about 1000 mg of the entactogen;
iii) the pharmaceutical composition comprising the NEI and the pharmaceutical composition comprising the entactogen are independently administered by a route independently selected from the group consisting of oral transdermal, transmucosal, (intra)nasal and (trans)rectal, intravesical, intrapulmonary, intraduodenal, intragastrical, intrathecal, subcutaneous, intramuscular, intradermal, intra-arterial, intravenous, intrabronchial, inhalation, and topical administration;
iv) the pharmaceutical composition comprising the NEI and the pharmaceutical composition comprising the entactogen each independently further comprise at least one pharmaceutically acceptable carrier or excipient; and
v) the single dosage form comprises an anti-abuse formulation.

7. (canceled)

8. The method of claim 1, wherein the pharmaceutical composition comprising the NEI and the pharmaceutical composition comprising the entactogen are formulated as separate dosage forms.

9. The method of claim 8, wherein the pharmaceutical composition comprising the NEI is administered to the subject about 1 minute to about 24 hours before the pharmaceutical composition comprising the entactogen is administered to the subject.

10. The method of claim 1, wherein the pharmaceutical composition comprising the NEI and the pharmaceutical composition comprising the entactogen are administered concurrently.

11. The method of claim 1, wherein the pharmaceutical composition comprising the NEI and the pharmaceutical composition comprising the entactogen are formulated as a single dosage form.

12-14. (canceled)

15. A method of treating or ameliorating post-traumatic stress disorder in a subject in need thereof, the method comprising:
administering to the subject:
i) a pharmaceutical composition comprising a therapeutically effective amount of a α_{2A}-adrenergic receptor agonist (AARA), or a pharmaceutically acceptable salt, solvate, enantiomer, diastereomer, or tautomer thereof; and
ii) a pharmaceutical composition comprising a therapeutically effective amount of a psychedelic agent or a pharmaceutically acceptable salt, solvate, enantiomer, diastereomer, or tautomer thereof,
wherein the pharmaceutical composition comprising the AARA and the pharmaceutical composition comprising the psychedelic agent are formulated as a single dosage form or as separate dosage forms; and
wherein the post-traumatic stress disorder in the subject is treated or ameliorated.

16. The method of claim 15, wherein the AARA is selected from the group consisting of clonidine, dexmedetomidine, fadolimidine, guanfacine, guanabenz, guanoxabenz, guanethidine, xylazine, tizanidine, methyldopa, methylnorepinephrine, norepinephrine, detomidine, and lofexidine, or a pharmaceutically acceptable salt, solvate, enantiomer, diastereomer, or tautomer thereof.

17. The method of claim 15, wherein the psychedelic agent is selected from the group consisting of psilocybin, 2C—B (4-bromo-2,5-dimethoxyphenethylamine), ketamine, DMT (N,N-dimethyltryptamine), 5-MeO-DMT (5-methoxy-dimethyltryptamine), LSA (d-lysergic acid amide), and LSD (lysergic acid diethylamide) or a pharmaceutically acceptable salt, solvate, enantiomer, diastereomer, or tautomer thereof.

18. The method of claim 16, wherein the AARA is guanfacine.

19. The method of claim 17, wherein the psychedelic agent is psilocybin.

20. The method of claim 15, wherein at least one of the following applies:
i) the subject is administered about 1 to about 1000 mg of the AARA;
ii) the subject is administered about 1 to about 1000 mg of the psychedelic agent;
iii) the pharmaceutical composition comprising the AARA and the pharmaceutical composition comprising the psychedelic agent are independently administered by a route independently selected from the group consisting of oral transdermal, transmucosal, (intra)nasal and (trans)rectal, intravesical, intrapulmonary, intraduodenal, intragastrical, intrathecal, subcutaneous, intramuscular, intradermal, intra-arterial, intravenous, intrabronchial, inhalation, and topical administration;
iv) the pharmaceutical composition comprising the NEI and the pharmaceutical composition comprising the entactogen each independently further comprise at least one pharmaceutically acceptable carrier or excipient; and
v) the single dosage form comprises an anti-abuse formulation.

21. (canceled)

22. The method of claim 15, wherein the pharmaceutical composition comprising the AARA and the pharmaceutical composition comprising the psychedelic agent are formulated as separate dosage forms.

23. The method of claim 22, wherein the pharmaceutical composition comprising the AARA is administered to the subject about 1 minute to about 24 hours before the pharmaceutical composition comprising the psychedelic agent is administered to the subject.

24. The method of claim 15, wherein the pharmaceutical composition comprising the AARA and the pharmaceutical composition comprising the psychedelic agent are administered concurrently.

25. The method of claim 15, wherein the pharmaceutical composition comprising the AARA and the pharmaceutical composition comprising the psychedelic agent are formulated as a single dosage form.

26-28. (canceled)
